(19) United States
(12) Patent Application Publication
(10) Pub. No.: US 2012/0148577 A1
(43) Pub. Date: Jun. 14, 2012

(54) USE OF HIGH-DOSE, POST-TRANSPLANTATION OXAZAPHOSPHORINE DRUGS FOR REDUCTION OF TRANSPLANT REJECTION

(75) Inventors: EPHRAIM J. FUCHS, Owings Mills, MD (US); LEO LUZNIK, Baltimore, MD (US); ROBERT A. BRODSKY, Brooklandville, MD (US); RICHARD J. JONES, Baltimore, MD (US); FRANCIS E. O'DONNELL, JR., Longboat Key, FL (US); CARLOS SANTOS, Tampa, FL (US); SUSAN BONITZ, Flemington, NJ (US)

(73) Assignees: ACCENTIA BIOPHARMACEUTICALS, INC., Tampa, FL (US); JOHNS HOPKINS UNIVERSITY, Baltimore, MD (US)

(21) Appl. No.: 13/272,864

(22) Filed: Oct. 13, 2011

Related U.S. Application Data
(63) Continuation of application No. 13/034,222, filed on Feb. 24, 2011, now abandoned.
Application No. 13/034,222 is a continuation of application No. 12/841,854, filed on Jul. 22, 2010, now abandoned.
Application No. 12/841,854 is a continuation of application No. PCT/US2009/031703, filed on Jan. 22, 2009.
(60) Provisional application No. 61/088,570, filed on Aug. 13, 2008.
(60) Provisional application No. 61/022,774, filed on Jan. 22, 2008.

(51) Int. Cl. A61K 31/664; A61K 35/14; A61K 35/18; A61K 31/7076; A61K 39/395; A61P 31/12; A61K 39/00; A61K 35/12; A61P 37/06; A61P 35/00; A61P 31/00; A61K 38/19; A61K 31/7056

(52) U.S. Cl. 424/133.1; 514/110; 424/85.1; 424/93.72; 424/93.73; 514/48; 424/130.1; 514/43; 514/46; 424/184.1; 424/93.7

(57) ABSTRACT

A lymphocytotoxic, but hematopoietic stem cell-sparing, high-dose amount of an oxazaphosphorine drug such as, for example, cyclophosphamide, administered post-transplantation can be used to reduce transplant rejection, including graft-versus-host-disease (GVHD). In some embodiments, the transplants are bone marrow transplants or hematopoietic stem cell transplants carried out for the treatment of hematologic disorders, including hematologic malignancies and non-malignant hematologic disorders. In some embodiments, the transplants are carried out for the treatment of hereditary hemoglobinopathies, such as sickle cell anemia and thalassemia.

### CROSS-REFERENCE TO RELATED APPLICATIONS

The present application is a continuation of U.S. patent application Ser. No. 13/034,222, filed Feb. 24, 2011, which is a continuation of U.S. patent application Ser. No. 12/841,854, filed Jul. 22, 2010, which is a continuation of International Application PCTXS2009/031703, filed Jan. 22, 2009, which claims the benefit of U.S. Provisional Application Ser. No. 61/022,774, filed Jan. 22, 2008 and U.S. Provisional Application Ser. No. 61/088,570, filed Aug. 13, 2008, each of which is hereby incorporated by reference herein in its entirety, including any figures, tables, nucleic acid sequences, amino acid sequences, and drawings.

### GOVERNMENT SUPPORT

This invention was made with government support under P01 CA15396 awarded by the National Cancer Institute. The government has certain rights in the invention.

### BACKGROUND OF THE INVENTION

Allogeneic blood or marrow transplantation (BMT) is potentially curative for a variety of life-threatening non-malignant hematologic diseases including sickle cell disease, aplastic anemia, paroxysmal nocturnal hemoglobinuria (PNH), thalassemia and others. The best results in sickle cell disease are with myeloablative conditioning regimens in children and young adults using human leukocyte antigen (HLA)—matched siblings (Panepinto, J. A. et al. (2007) Br. J. Haematol., 137, 479-485; Bernaudin, F. et al. (2007) Blood, 110, 2749-2756). However, PNH and sickle cell patients often have significant end-organ toxicities that disqualify them from myeloablative BMT. Reduced intensity BMT from matched siblings has been successful in PNH (Takahashi, Y. et al. (2004) Blood, 103, 1383-1390) but graft rejection has been a major obstacle to this approach in sickle cell disease (Van, B. K. et al. (2000) Bone Marrow Transplant., 26, 445-449; Iannone, R. et al. (2003) Biol. Blood Marrow Transplant., 9, 519-528; Horan, J. T. et al. (2005) Bone Marrow Transplant., 35, 171-177).

Identifying a suitable donor also limits the application of BMT, especially for sickle cell anemia; fewer than 18% of patients with sickle cell disease have a suitable HLA-matched sibling donor (Mentzer, W. C. et al. (1994) Am. J. Pediatr. Hematol. Oncol., 16, 27-29). In order to expand the potential donor pool, umbilical cord blood BMT has been tried for sickle cell disease; however, the high incidence of graft failure has also limited the enthusiasm for this approach (Adamkiewicz, T. V. et al. (2007) Pediatr. Transplant., 11, 641-644; Walters, M. C. (2007) Pediatr. Transplant., 11, 582-583).

Related haploidentical BMT is an alternative method for expanding the potential pool of bone marrow donors; any patient shares exactly one HLA haplotype with each biologic parent or child, and siblings or half-siblings have a 50% chance of being haploidentical. The disadvantage of this approach has been the high incidence of graft rejection and severe graft-versus-host disease (GVHD).

It has been previously demonstrated that high-dose cyclophosphamide has the potential to eradicate both autoimmunity (Brodsky, R. A. et al. (1996) Blood, 87, 491-494; Brodsky, R. A. et al. (1998) Annals of Internal Medicine, 129, 1031-1035; Brodsky, R. A. et (2001b) Annals of Internal Medicine, 135, 477-483) and alloimmunization (Brodsky, R. A. et al. (2001a) Transplantation, 71, 482-484). High-dose cyclophosphamide is not toxic to primitive hematopoietic stem cells because they possess high levels of aldehyde dehydrogenase, an enzyme that confers resistance to the drug (Hilton, J. (1984) Cancer Research, 44, 5156-5160; Jones, R. J. et al. (1995) Blood, 85, 2742-2746). Thus, high-dose cyclophosphamide is highly immunosuppressive, but not myeloablative. Recently, it has been shown that haploidentical BMT using non-myeloablative conditioning and high-dose, post-transplantation cyclophosphamide is associated with low rates of fatal graft failure, infection, and severe acute GVHD in patients with hematologic malignancies (O'Donnell, P. V. et al. (2006) Blood, 108, 894a-895a).

### BRIEF SUMMARY OF THE INVENTION

The present invention is based, at least in part, on the discovery that a lymphocytotoxic but hematopoietic stem cell-sparing high-dose pulsed amount of an oxazaphosphorine drug such as, for example, cyclophosphamide, can be used to reduce transplant rejection, including graft-versus-host-disease (GVHD). In some embodiments, the transplants are bone marrow transplants or hematopoietic stem cell transplants carried out for the treatment of hematologic disorders, including hematologic malignancies, as well as non-malignant hematological disorders. In some embodiments, the non-malignant hematological disorder is a hereditary hemoglobinopathy, such as sickle cell anemia and thalassemia.

In one aspect of the present invention, a method for reducing transplant rejection in a human or non-human mammal subject is provided. The method includes administering to the subject in need thereof, a lymphocytotoxic but hematopoietic stem cell-sparing high-dose pulsed amount of an oxazaphosphorine drug after transplantation (post-transplant), such that the subject's immune system reconstitutes (without the necessity of stein cell transplantation for such reconstitution), thereby reducing transplant rejection in the subject.

Examples of transplant rejections that may be treated reduced (e.g., alleviated or avoided) include, for example, transplant rejection occurring during or following allogeneic transplantation of organs, tissues, or cells into a subject (host); transplant rejection occurring during or following a xenogenic transplantation of organs, tissues, or cells into a host; and transplant rejection occurring during or following transplantation of autologous tissue, organs or cells into a host. The transplanted cells can range in plasticity from totipotent or pluripotent stem cells (e.g., adult or embryonic), precursor or progenitor cells, to highly specialized cells, such as those of the central nervous system (e.g., neurons and glia). Suitable cells can be selected by those of skill in the art as desired, e.g., for treatment of the underlying condition.

In some embodiments, the transplant cells are bone marrow cells, hematopoietic stem cells or hematopoietic progenitor cells, or other stem cells or progenitor cells. The transplant cells may be administered to the subject in an enriched (e.g., purified or isolated) or non-enriched form. Transplant stem and progenitor cells can be obtained from a variety of sources, including embryonic tissue, fetal tissue, adult tissue, umbilical cord blood, peripheral blood, bone marrow, and brain, for example.

As will be understood by one of skill in the art, there are over 200 cell types in the human body. It is believed that the methods of the subject invention can be used to reduce immunological rejection of any of these cell types upon transplantation. For example, any cell arising from the ectoderm, mesoderm, or endoderm germ cell layers can potentially be transplanted. In one embodiment, the transplanted cells are bone marrow cells. In another embodiment, the transplant is an allogeneic bone marrow transplant.

It will be understood by one of skill in the art that the methods of the present invention are also applicable for veterinary purposes. Cells of non-human animals can find application either in human or animal subjects (transplant recipients). For example, although dopamine neurons from human, pig, and rat are similar in that they synthesize dopamine and release synaptically into the brain, they differ immunologically, in extent of reinervation of the brain, in life span, and in infectious agents associated with the specific donor or donor species. These traits can be exploited for their specific strengths and weaknesses.

Transplant rejections also include rejections occurring during or following transplantation of an organ, tissue or cells from related (matched or partially matched) or unrelated donors (non-matched). Transplant rejections after transplantation include both graft rejection and graft-versus-host disease. Without wishing to be hound by theory, it is contemplated that any transplant rejection that can be effectively treated, delayed, or partially or completely avoided, by eliminating the subject's circulating immune cells (along with any donor lymphocytes in the transplant) with high dose cyclophosphamide and allowing them to redevelop from hematopoietic stem cells is encompassed by this disclosure.

As indicated above, in some embodiments, the transplantation is carried out for the treatment of hematologic disorders, including hematologic malignancies (malignant hematologic disorders) as well as non-malignant hematologic disorders such as hereditary hemoglobinopathies (e.g., sickle cell anemia and thalassemia). When the disorder to be treated by transplantation is a hematologic disorder, the transplants are typically bone marrow transplants or hematopoietic stem cell transplants. The hematologic disorder may be acquired or congenital. Examples of acquired hematologic disorders include, but are not limited to, acute lymphoblastic leukemia, acute lymphocytic leukemia, acute myelogenous leukemia, aplastic anemia, chronic myelogenous leukemia (accelerated phase or blast crisis), desmoplastic small round cell tumor, Ewing's sarcoma, Hodgkin's disease, multiple myeloma (Kahler's disease), myelodysplasia, non-Hodgkin's lymphoma, paroxysmal nocturnal hemoglobinuria (PNH); severe aplasia), radiation poisoning, chronic lymphocytic leukemia, AL amyloidosis, essential thrombocytosis, and polycythemia vera. Examples of inherited hematologic disorders include congenital adrenoleukodystrophy, amegakaryocytic thrombocytopenia, sickle cell disease, Griscelli syndrome type II, Hurler syndrome, Kostmann syndrome, Krabbe disease, metachromatic leukodystrophy, thalassemia, hemophagocytic lymphohistiocytosis (HLH), Wiskott-Aldrich syndrome, and Neuroblastoma. The disorder to be treated may be a coagulation disease, such as Bernard-Soulier syndrome, Glanzmann's thrombasthenia, grey platelet syndrome, protein C deficiency, protein S deficiency, antithrombin III deficiency, antiphospholipid syndrome, factor V Leiden. Other disorders to be treated include purpura, such as Henoch-Schönlein, ITP (Evans syndrome), thrombotic thrombocytopenic purpura. In addition to thalassemia and sickle-cell disease/trait, other examples of anemias/hemoglobinopathies to be treated included, G6PD Deficiency, hereditary spherocytosis, hereditary elliptocytosis, iron deficiency anemia, Plummer-Vinson syndrome, megaloblastic anemia (Pernicious anemia). Other hematologic disorders that may be treated include acute monocytic leukemia, malignant histiocytosis, Erdheim-Chester disease Asplenia/hyposplenism, Methemoglobinemia, WHO-I Langerhans cell histiocytosis, non-Langerhans-cell histiocytosis/WHO-II (Juvenile xanthogranuloma, Hemophagocytic lymphohistiocytosis), and malignant histiocytic disorders/WHO-III (e.g., acute monocytic leukemia, Malignant histiocytosis, Erdheim-Chester disease). In one embodiment, the subject is not suffering from a hematologic malignancy or other cancer.

The subject (transplant recipient) may or may not be suffering from an autoimmune disease. In some embodiments, the subject is not suffering from an autoimmune disease. Exemplary autoimmune diseases include, but are not limited to, AIDS-associated myopathy, AIDS-associated neuropathy, Acute disseminated encephalomyelitis, Addison's Disease, Alopecia Areata, Anaphylaxis Reactions, Ankylosing Spondylitis, Antibody-related Neuropathies, Antiphospholipid Syndrome, Autism, Autoimmune Atherosclerosis, Autoimmune Diabetes Insipidus, Autoimmune Endometriosis, Autoimmune Eye Diseases, Autoimmune Gastritis, Autoimmune Hemolytic Anemia, Autoimmune Hemophilia, Autoimmune Hepatitis, Autoimmune Interstitial Cystitis, Autoimmune Lymphoproliferative Syndrome, Autoimmune Myelopathy, Autoimmune Myocarditis, Autoimmune Neuropathies, Autoimmune Oophoritis, Autoimmune Orchitis, Autoimmune Thrombocytopenia, Autoimmune Thyroid Diseases, Autoimmune Urticaria, Autoimmune Uveitis, Autoimmune Vasculitis, Behcet's Disease, Bell's Palsy, Bullous Pemphigoid, CREST, Celiac Disease, Cerebellar degeneration (paraneoplastic), Chronic Fatigue Syndrome, Chronic Rhinosinusitis, Chronic inflammatory demyelinating polyneuropathy, Churg Strauss Syndrome, Connective Tissue Diseases, Crohn's Disease, Cutaneous Lupus, Dermatitis Herpetiformis, Dermatomyositis, Diabetes Mellitus, Discoid Lupus Erythematosus, Drug-induced Lupus, Endocrine Orbitopathy, Glomerulonephritis, Goodpasture Syndrome, Goodpasture's Syndrome, Graves Disease, Guillian-Barre Syndrome, Miller Fisher variant of the Guillian Barre Syndrome, axonal Guillian Barre Syndrome, demyelinating Guillian Bane Syndrome, Hashimoto Thyroiditis, Herpes Gestationis, Human T-cell lymphomavirus-associated myelopathy, Huntington's Disease, IgA Nephropathy, Immune Thrombocytopenic Purpura, Inclusion body myositis, Interstitial Cystitis, Isaacs syndrome, Lambert Eaton myasthenic syndrome, Limbic encephalitis, Lower motor neuron disease, Lyme Disease, MCTD, Microscopic Polyangiitis, Miller Fisher Syndrome, Mixed Connective Tissue Disease, Mononeuritis multiplex (vasculitis), Multiple Sclerosis, Myasthenia Gravis, Myxedema, Meniere Disease, Neonatal LE, Neuropathies with dysproteinemias, Opsoclonus-myoclonus, PBC, POEMS syndrome, Paraneoplastic Autoimmune Syndromes, Pemphigus, Pemphigus Foliaceus, Pemphigus Vulgaris, Pernicious Anemia, Peyronie's Disease, Plasmacytoma/myeloma neuropathy, Poly-Dermatomyositis, Polyarteritis Nodosa, Polyendocrine Deficiency Syndrome, Polyendocrine Deficiency Syndrome Type 1, Polyendocrine Deficiency Syndrome Type 2, Polyglandular Autoimmune Syndrome Type I, Polyglandular Autoimmune Syndrome Type II, Polyglandular Autoimmune Syndrome Type III, Polymyositis, Primary Biliary Cirrhosis, Primary Glomerulonephritis, Primary Sclerosing Cholangitis, Psoriasis, Psoriatic Arthritis, Rasmussen's Encephalitis, Raynaud's Disease, Relapsing Polychondritis, Retrobulbar neuritis, Rheumatic Diseases, Rheumatoid Arthritis, Scleroderma, Sensory neuropathies (paraneoplastic), Sjogren's Syndrome, Stiff-Person Syndrome, Subacute Thyroiditis, Subacute autonomic neuropathy, Sydenham Chorea, Sympathetic Ophthalmitis, Systemic Lupus Erythematosus, Transverse myelitis, Type 1 Diabetes, Ulcerative Colitis, Vasculitis, Vitiligo, Wegener's Granulomatosis, Acrocyanosis, Anaphylactic reaction, Autoimmune inner ear disease, Bilateral sensorineural hearing loss, Cold agglutinin hemolytic anemia, Cold-induced immune hemolytic anemia, Idiopathic endolymphatic hydrops, Idiopathic progressive bilateral sensorineural hearing loss, Immune-mediated inner ear disease, and Mixed autoimmune hemolysis.

The lymphocytotoxic, non-myeloablative amount of oxazaphosphorine drug (e.g., cyclophosphamide) is administered post-transplantation. However, in embodiments in which a pre-transplant conditioning regimen is employed, an oxazaphosphorine such as cyclophosphamide and/or one or more other immunosuppressive agents or treatments may also be administered to the subject before transplantation, such as total body irradiation, total lymph node irradiation, radiolabeled antibody against leukocytes, fludarabine, anti-thymocyte globulin (ATG), pentostatin, 2-chlorodeoxyadenosine (2CdA), fludarabine-like drug, campath (alemtuzumab), busulfan, melphalan, chlorambucil, and uramustine.

In some embodiments, the lymphocytotoxic, non-myeloablative amount of oxazaphosphorine drug is in the range of about 40 mg/kg/day to about 60 mg/kg/day (e.g., about 50 mg/kg/day) for one or more consecutive days post-transplantation. Preferably, the lymphocytotoxic non-myeloablative amount of the oxazaphosphorine drug is administered for 1-4 consecutive days. In some embodiments, the lymphocytotoxic non-myeloablative amount of oxazaphosphorine drug is administered for two or more consecutive days. Preferably, the lymphocytotoxic non-myeloablative amount of oxazaphosphorine drug is administered starting 48-72 hours after transplantation, for 1-4 consecutive days. In some embodiments, the lymphocytotoxic non-myeloablative amount of oxazaphosphorine drug is administered starting at least 60 hours after transplantation. In some embodiments, the lymphocytotoxic non-myeloablative amount of oxazaphosphorine drug is administered on day+3 and day+4 after transplantation.

In some embodiments, the lymphocytotoxic non-myeloablative amount of oxazaphosphorine drug is 50 mg/kg/day, administered for 1-4 consecutive days. In some embodiments, the lymphocytotoxic non-myeloablative amount of oxazaphosphorine drug is 50 mg/kg/day, administered for two or more consecutive days.

Optionally, the cells to be transplanted can be pre-treated with an oxazaphosphorine drug prior to transplantation. In this case, the subject is also preferably administered the oxazaphosphorine drug directly, (i) before transplantation, (ii) during transplantation, (iii) after transplantation, or any combination of two or more of the foregoing.

Another aspect of the invention pertains to transplant cells (e.g., cells, tissue, or organ) incubated or otherwise treated (contacted) with an oxazaphosphorine drug, such as cyclophosphamide.

Optionally, radiation therapy (e.g., total body irradiation (TBI) or total lymphoid irradiation) is also carried out on the subject.

Exemplary oxazaphosphorine drugs include, but are not limited to, cyclophosphamide, ifosfamide, perfosfamide, trophosphamide (trofosfamide), or a pharmaceutically acceptable salt, solvate, prodrug and metabolite thereof. In some embodiments, an oxazaphosphorine drug used in the methods described herein is cyclophosphamide or a pharmaceutically acceptable salt or metabolite thereof.

Also encompassed by this disclosure is a kit for reducing transplant rejection, including: (a) a plurality of doses of a lymphocytotoxic non-myeloablative but hematopoetic cell-sparing high-dose pulsed amount of a oxazaphosphorine drug, e.g., cyclophosphamide; (b) cells for transplantation; and (c) instructions for treating or avoiding transplant rejection using one or more doses of the oxazaphosphorine drug, wherein the transplant rejection is treated or avoided. The kit may further include one or more immunosuppressive agents, such as oxazaphosphorine, fludarabine, anti-thymocyte globulin (ATG), pentostatin, 2-chlorodeoxyadenosine (2CdA), fludarabine-like drug, campath (alemtuzumab), busulfan, melphalan, chlorambucil, and uramustine.

### BRIEF DESCRIPTION OF THE DRAWINGS

FIG. 1 shows the treatment schema used for patients 1-3 in Example 4, including conditioning therapy and post-transplant GVHD immunosuppression. Cy=cyclophosphamide; MMF=mycophenolate mofitil; TBI=total body irradiation.

FIG. 2 is a graph showing neutrophil recovery of patients 1 and 3 after haplo-identical BMT.

FIG. 3 is a dual color display of peripheral blood granulocytes from patients 1 and 3 after staining with anti-CD15 PE and FLAER. Granulocytes were analyzed before (Pre) and at days 15, 30, and 360, after blood or marrow transplantation (BMT).

FIG. 4 is a graph showing hemoglobin (Hb) variants from patient 3 before and after BMT.

### DETAILED DESCRIPTION OF THE INVENTION

The present invention is based, at least in part, on the discovery that administration of a lymphocytotoxic non-myeloablative amount of an oxazaphosphorine drug can be used for replacing a subject's immune cells, including lymphocytes and immune cells associated with immune tolerance, with disease-free immune cells, without the need for stem cell transplantation. The rationale underlying this approach is the discovery that oxazaphosphorine drugs such as cyclophosphamide are lymphocytotoxic but spare hematopoietic progenitor stem cells because of high levels of aldehyde dehydrogenase, an enzyme which confers resistance to cyclophosphamide. The contents of U.S. patent application publication no. US 2007/0202077, entitled “Use of High-Dose Oxazaphosphorine Drugs for Treating Immune Disorders” are incorporated herein by reference in its entirety. Furthermore, high-dose oxazaphosphorines such as cyclophosphamide are effective as a single agent strategy for limiting acute and chronic Graft-Versus-Host-Disease (GVHD) after myeloablative HLA-matched related and unrelated allografting; this approach also limits the need for prolonged immunosuppression, resulting in favorable immunoreconstitution with few opportunistic infections in this unfavorable group of patients.

GVHD is a particularly severe type of transplant rejection characterized by the donor marrow (graft) producing immune cells that attack multiple organs of the recipient (host). Graft-versus-host disease is a common complication of allogeneic bone marrow transplantation in which functional immune cells in the transplanted marrow recognize the recipient as “foreign” and mount an immunologic attack. Rejection is frequent in bone marrow transplants, as the transplanted immune system from the donor attacks the organs of the transplant recipient, and many life-threatening complications occur. Using high-dose oxazaphosphorine treatment can improve the percentage of successful transplants, e.g., reducing the incidence of chronic GVHD in transplants (e.g., allogeneic blood or bone marrow transplants). After bone marrow transplantation, T cells present in the graft, either as contaminants or intentionally introduced into the host, attack the tissues of the transplant recipient after perceiving host tissues as antigenically foreign. The T cells produce an excess of cytokines, including TNF alpha and interferon-gamma (IFNγ). A wide range of host antigens can initiate graft-versus-host disease, among them the human leukocyte antigens (HLAs). However, graft-versus-host disease can occur even when HLA-identical siblings are the donors.

While donor T cells are undesirable as effector cells of graft-versus-host-disease, they are valuable for engraftment by preventing the recipient's residual immune system from rejecting the bone marrow graft (host-versus-graft). Additionally, as bone marrow transplantation is frequently used to cure cancer, mainly leukemias, donor T-cells have proven to have a valuable graft-versus-tumor effect.

As a prodrug, cyclophosphamide is converted to 4-hydroxycyclophosphamide and its tautomer aldophosphamide in the liver. These compounds diffuse into cells and are converted into the active compound phosphoramide mustard. Alternatively, they are inactivated by the enzyme aldehyde dehydrogenase to form the inert carboxyphosphamide. Lymphoid cells, including NK cells, and B and T lymphocytes, have low levels of aldehyde dehydrogenase and are rapidly killed by high doses (i.e., lymphocytotoxic) of cyclophosphamide In contrast, hematopoietic progenitor stem cells possess high levels of aldehyde dehydrogenase, rendering them resistant to cyclophosphamide. (See, for example, Hilton, Cancer Res., 44:5156-60 (1984); Kastan et al., Blood, 75:1947-50 (1990); Zoumbos et al., N. Eng. J. Med., 312:257-265 (1985); Brodsky, Sci. World J., 2: 1808-15 (2002)).

The present invention provides a method of reducing transplant rejection in a mammalian subject, including administering to the subject, post-transplant, a lymphocytotoxic but hematopoietic cell-sparing amount of an oxazaphosphorine drug, such that the subject's immune system reconstitutes, thereby reducing immunological rejection of the transplant. The subject's immune system reconstitutes without the need for stem cell transplantation; nonetheless, the transplant may include stem cells for treatment of an underlying disorder, such as a hematologic disorder (e.g., hematologic malignancy, or non-malignant hematologic disorder such as sickle cell anemia).

As will be understood by those skilled in the art, there are over 200 cell types in the human body. The methods of the subject invention are useful in treating or preventing immunologic rejection of any of these cell types. For example, transplant cells can include those cells arising from the ectoderm, mesoderm, or endoderm germ cell layers. Such cells include, but are not limited to, bone marrow cells, neurons, glial cells (astrocytes and oligodendrocytes), muscle cells (e.g., cardiac, skeletal), chondrocytes, fibroblasts, melanocytes, Langerhans cells, keratinocytes, endothelial cells, epithelial cells, pigment cells (e.g., melanocytes, retinal pigment epithelial (RPE) cells, iris pigment epithelial (IPE) cells), hepatocytes, microvascular cells, pericytes (Rouget cells), blood cells (e.g., erythrocytes), cells of the immune system (e.g., B and T lymphocytes, plasma cells, macrophages/monocytes, dendritic cells, neutrophils, eosinophils, mast cells), thyroid cells, parathyroid cells, pituitary cells, pancreatic cells (e.g., insulin-producing beta cells, glucagon-producing alpha cells, somatostatin-producing delta cells, pancreatic polypeptide-producing cells, pancreatic ductal cells), stromal cells, adipocytes, reticular cells, rod cells, and hair cells. Other examples of cell types that can be transplanted include those disclosed by Spier R. E. et al., eds., (2000) The Encyclopedia of Cell Technology, John Wiley & Sons, Inc., and Alberts B. et al., eds., (1994) Molecular Biology of the Cell, 3^{rd} ed., Garland Publishing, Inc., e.g., pages 1188-1189.

Various cell lines have also been used in animal models of transplantation for a variety of purposes. Fetal kidney cells and amniotic cells have been transplanted as sources of trophic factors. Adrenal medullary cells, sympathetic ganglion cells, and carotid body cells have been transplanted as sources of dopamine. Fibroblasts and glial cells have been transplanted as sources of trophic factors, to carry genes through recombinant strategies, or for demyelinating diseases, for example. Corneal endothelial cells have been used for corneal transplants. Myoblasts have been transplanted for the treatment of muscular dystrophy and cardiac disease. Other cell lines include pancreatic islet cells for diabetes; thyroid cells for thyroid disorders; blood cells for AIDS, bone marrow transplant, and inherited disorders; bone and cartilage for osteoarthritis, rheumatoid arthritis, or for fracture repair; skin or fat cells for reconstructive purposes, such as in skin grafts after burns or cosmetic surgery; breast augmentation with fat; hair follicle replacement; liver cells for liver disorders inducing hepatitis; and retinal pigment epithelial cells (RPE) for retinitis pigmentosa and Parkinson's disease.

Stem cells are believed to have immense potential for therapeutic purposes for numerous diseases. Stem cells have been derived from numerous donor sources, including, but not limited to, embryonic, blast, tissue-derived, blood, and cord-blood cells; organ-derived progenitor cells; and bone marrow stromal cells, among others. Such stem cells can be differentiated along numerous pathways to produce virtually any cell type. These cells can be transplanted either before or after differentiation. Hematopoietic stem cells (HSC) have been used for many years, and are used typically for treatment of hematopoietic cancers (e.g., leukemias and lymphomas), and non-hematopoietic malignancies (cancers in other organs). Other indications include diseases that involve genetic or acquired bone marrow failure, such as aplastic anemia, thalassemia, sickle cell anemia, and autoimmune diseases. HSC for transplantation can be obtained, for example, from bone marrow, peripheral blood, or umbilical cord blood.

Methods and markers commonly used to identify stem cells and to characterize differentiated cell types are described in the scientific literature (e.g., Stem Cells: Scientific Progress and Future Research Directions, Appendix E1-E5, report prepared by the National Institutes of Health, June, 2001). The list of adult tissues reported to contain stem cells is growing and includes bone marrow, peripheral blood, umbilical cord blood, brain, spinal cord, dental pulp, blood vessels, skeletal muscle, epithelia of the skin and digestive system, cornea, retina, liver, and pancreas.

In some embodiments of the present invention, additional agents and in particular agents which facilitate transplant cell growth are preferably administered to the subject following the administration of a lymphocytotoxic but hematopoietic stem-cell sparing high-dose pulsed amount of an oxazaphosphorine drug such as, for example, cyclophosphamide.

For example, in the case of blood or bone marrow transplantation, agents that facilitate hematopoietic stem cell growth such as, filgrastim and pegfilgrastin, can be administered to the subject following the administration of a lymphocytotoxic but hematopoietic stem-cell sparing high-dose pulsed amount of an oxazaphosphorine drug (e.g., cyclophosphamide).

### I. Definitions

In order that the present disclosure may be more readily understood, certain terms are first defined. Additional definitions are set forth throughout the detailed description.

As used herein, the phrase “high-dose pulsed amount of an oxazaphosphorine drug” refers to a non-myeloablative amount of an oxazaphosphorine drug such as, for example, cyclophosphamide, which is immunosuppressive, upon single or multiple dose administration to a subject (such as a human patient undergoing transplantation), thereby resulting in a substantial reduction in or complete elimination of mature circulating lymphocytes in the subject (as well as mature donor lymphocytes in the transplant). In some embodiments, administration of a non-myeloablative amount of cyclophosphamide results in treating, preventing, curing, delaying, reducing the severity of, ameliorating at least one symptom of transplant rejection, or prolonging the survival of the subject beyond that expected in the absence of such administration. In some embodiments, “high-dose pulsed amount of an oxazaphosphorine drug” refers to a dose of cyclophosphamide administered to a subject in need thereof, which results in eliminating or substantially reducing the number of circulating lymphocytes in the subject, including those which are associated with immune tolerance, while sparing the hematopoietic progenitor stem cells. For example, in some embodiments, “high-dose pulsed amount of an oxazaphosphorine drug” is a 50 mg/kg/day dose of an oxazaphosphorine drug such as, for example, cyclophosphamide, administered to a subject post-transplant, for 1-4 days. Cyclophosphamide is sold under common trade-names including PROCYTOX, CYTOXAN and NEOSAR.

The term “reducing”, in the context of reducing transplant rejection, is intended to be inclusive of reducing the severity of transplant rejection, eliminating or reducing the severity of one or more clinical symptoms of transplant rejection, eliminating transplant rejection, preventing onset of transplant rejection, or delaying onset of transplant rejection. Thus, the term “reducing”, includes therapeutic and preventative measures.

The terms “treating,” and “treatment,” as used herein, refer to therapeutic or preventative measures described herein. The methods of “treatment” employ administration to a subject (transplant recipient or prospective transplant recipient), a lymphocytotoxic non-myeloablative amount of an oxazaphosphorine drug such as, for example, cyclophosphamide in order to prevent, cure, delay onset, reduce the severity of, or ameliorate one or more symptoms of transplant rejection (including graft rejection or graft-versus-host disease (GVHD), e.g., acute or chronic)) or in order to prolong the survival of a subject beyond that expected in the absence of such treatment. Criteria for the development of graft rejection and GVHD are known in the art. For example, acute GVHD may be graded clinically according to the criteria developed by the consensus conference on acute GVHD (Przepiorka D. et al., Consensus Conference on Acute GVHD Grading, Bone Marrow Transplant, 15:825-828 (1995)).

The terms “cure” and “curing,” as used herein, refer to a remission of a disease or an elimination of symptoms (e.g., clinical, laboratory, and imaging) of a disease in a subject such as, for example, transplant rejection, by the methods described herein. The remission of a disease or the elimination of symptoms of a disease in a subject may be for at least about 1 year, at least about 2 years, at least about 3 years, at least about 4 years, or at least about 5 years. In certain embodiments, a remission of a disease or an elimination of symptoms of a disease in a subject includes the absence of administering alternative methods of treatment.

The term “hematopoietic progenitor stem cell,” as used herein refers to any type of cell of the hematopoietic system, including, but not limited to, undifferentiated cells such as hematopoietic stem cells and progenitor cells, which are capable of reconstituting the immune system following administration of a lymphocytotoxic non-myeloablative amount of cyclophosphamide to a subject identified using the methods described herein. In some embodiments of the methods, compositions, transplants, and kits of the invention, the transplant cells comprise hematopoietic progenitor stem cells.

The terms “B lymphocyte” and “B cell,” as used interchangeably herein, are intended to refer to any cell within the B cell lineage as early as B cell precursors, such as pre-B cells B220⁺ cells which have begun to rearrange Ig VH genes and up to mature B cells and including plasma cells. Such cells can be readily identified by one of ordinary skill in the art using standard techniques known in the art and those described herein.

The terms “immunoablation” and “immunoablative,” as used herein, refer to severe immunosuppression using a high-dose (i.e., lymphocytotoxic non-myeloablative amount) of cyclophosphamide, for example, 50 mg/kg X 4 days of cyclophosphamide, which leads to substantial reduction in or elimination of the population of circulating lymphocytes, including for example, NK cells and B and T lymphocytes. Immunoablation, as described herein, results in complete or substantially complete reduction in immune cells responsible for immune tolerance.

The term “lymphocytotoxic,” as used herein, refers to complete elimination of or substantial reduction in the number of circulating lymphocytes, including those associated with immune tolerance in a subject following administration of a high-dose (i.e., lymphocytotoxic non-myeloablative amount) of an oxazaphosphorine drug, such as, for example, 50 mg/kg X 4 days of cyclophosphamide. Substantial reduction can be a reduction of about 75%, 90%, 95%, 98%, 99% of the circulating lymphocytes. The term “lymphocytotoxic,” includes killing of those immune cells by cyclophosphamide which express low levels of the enzyme aldehyde dehydrogenase.

The term “non-myeloablative,” as used herein, refers to a property of a compound such as, for example, an oxazaphosphorine drug such as cyclophosphamide, whereby the compound does not have a cytotoxic effect on myeloid stem cells, for example, hematopoietic progenitor stem cells. In some embodiments, a non-myeloablative agent used in the methods described herein has a cytotoxic effect on the circulating mature lymphocytes (e.g., NK cells, and T and B lymphocytes) while sparing the progenitor cells, e.g., hematopoietic progenitor stem cells that are capable of reconstituting the immune system. In some embodiments, a non-myeloablative agent used in the methods of the invention kills cells which express low levels of the enzyme aldehyde dehydrogenase (e.g., NK cells and B and T lymphocytes) while sparing cells which express high or resistant levels of the enzyme aldehyde dehydrogenase (e.g., hematopoietic progenitor stem cells).

The term “immunomodulatory agent,” as used herein, refers to agents other than a oxazaphosphorine drug, which are capable of modulating the immune system (e.g., by increasing or decreasing an immune response; increasing or decreasing activity of one or more immune cells and/or activating or suppressing the immune system), in the methods described herein. For example, in some embodiments, immunomodulatory agents include immunosuppressive agents, other than a oxazaphosphorine agent such as cyclophosphamide, which when administered at an appropriate dosage, results in the inhibition of an immune response, for example, inhibition of T cell activity. Examples of such agents include, but are not limited to, prednisone, cyclosporine, tacrolimus, mycophenolate mofetil, and rapamycin. In some embodiments, exclusion of any additional immunomodulatory agents in methods described herein, refers to exclusion of additional immunosuppressive agents subsequent to, or concurrently with the administration of a lymphocytotoxic non-myeloablative amount of a oxazaphosphorine drug.

Methods which do not include the use of “any additional immunomodulatory agents” can specifically exclude the use of agents which are immunosuppressive, such as, for example, prednisone, in methods which use a lymphocytotoxic non-myeloablative amount of an oxazaphosphorine drug.

The term “transplantation”, as used herein, includes the administration of cells that have been grown in vitro, and may have been genetically modified, as well as the transplantation of material extracted from subject or another organism. Cells may be administered by any of a variety of routes that is effective in delivering the transplant cells to the target site or sites. Methods for transplantation of cells into humans and animals are known to those in the art and are described in the literature in the art. Cells may be administered with a pharmaceutically acceptable carrier. The transplantation may be carried out to treat a disease or disorder such as a hematologic disorder (e.g., hematologic malignancies or hereditary hemoglobinopathies (e.g., siclde cell anemia)), or as part of reparative, reconstruction, or elective surgery. Depending upon the source of the cells to be transplanted, transplantation may be autologous, allogeneic, xenogenic, or a combination of two or more of the foregoing (e.g., autologous cells and allogeneic cells).

The term “cells” in the context of transplantation (transplant cells) is intended to refer to any of among cells in isolation (isolated cells), a tissue, and an organ. Thus, transplantation “cells” is inclusive of transplantation of a tissue or organ. In some embodiments, transplantation of cells may be carried out with purified cells, non-purified, mixed cell populations (such as mobilized peripheral blood, cord blood, or bone marrow), or cell populations that have been enriched for a particular cell type or cell types but have not been fully purified. For example, HSCs may be enriched through column selection for CD34⁺ cells. Cells may be genetically modified or non-genetically modified. The transplanted cells may be autologous, allogeneic, or xenogenic to the recipient (host). In one embodiment, the transplanted cells are bone marrow cells, such as allogeneic bone marrow cells.

As used herein, the term “genetically modified” refers to cells that have been manipulated to contain a non-native (heterologous) polynucleotide (e.g., a transgene) by recombinant methods. For example, cells can be genetically modified by introducing a nucleic acid molecule that encodes a selected polypeptide.

As used herein, the term “transgene” refers to a polynucleotide (e.g., DNA or RNA) that is inserted into a cell and that encodes an amino acid sequence corresponding to a functional protein. Optionally, the encoded protein is capable of exerting a therapeutic or regulatory effect.

As used herein, the terms “protein” or “polypeptide” includes proteins, functional fragments of proteins, and peptides, whether isolated from natural sources, produced by recombinant techniques or chemically synthesized. Typically, the polypeptides typically comprise at least about 6 amino acids, and are preferably sufficiently long to exert a biological or therapeutic effect.

As used herein, the term “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, the term “expression control sequence” means a nucleic acid sequence that directs transcription of a nucleic acid. An expression control sequence can he a promoter, such as a constitutive or an inducible promoter, or an enhancer. The expression control sequence is operably linked to the nucleic acid sequence to be transcribed.

As used herein, the term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally-occurring nucleotides.

As used herein, the term “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

The terms “a” and “an” are intended to mean “at least one” unless clearly indicated otherwise. For example, reference to an oxazaphosphorine drug includes a plurality of such drugs. Reference to a cell includes a plurality of cells. Reference to an immunosuppressive agent includes a plurality of immunosuppressive agents, and so forth.

### II. Exemplary Oxazaphosphorine Drugs

The present invention provides methods of reducing transplant rejection using a lymphocytotoxic but hematopoietic stem cell-sparing amount of an oxazaphosphorine drug post-transplant. Exemplary oxazaphosphorine drugs that may be used in the methods of the invention include, but are not limited to, for example, cyclophosphamide (CPA), ifosfamide (IFO), and trofosfamide, perfosfamide, or a pharmaceutically acceptable salt, solvate, prodrug and active metabolite thereof. Active metabolites are those metabolites that retaining alkylating activity. CPA is widely used in low to intermediate amounts as an anticancer drug, an immunosuppressant, and for the mobilization of hematopoietic progenitor cells from the bone marrow into peripheral blood prior to bone marrow transplantation for aplastic anemia, leukemia, and other malignancies. Additional oxazaphosphorine drugs that may be used in the methods of the invention include, for example, mafosfamide (NSC 345842), glufosfamide (D19575, beta-D-glucosyl isophosphoramide mustard), NSC 612567 (aldophosphamide perhydrothiazine), and NSC 613060 (aldophosphamide thiazolidine).

Both CPA and IFO are prodrugs that require activation by hepatic cytochrome P450 (CYP)-catalyzed 4-hydroxylation, yielding cytotoxic nitrogen mustards capable of reacting with DNA molecules to form crosslinks and lead to cell apoptosis and/or necrosis. However, more newly synthesized oxazaphosphorine derivatives such as glufosfamide, NSC 612567 and NSC 613060, do not need hepatic activation. They are activated through other enzymatic and/or non-enzymatic pathways.

In some embodiments according to the present invention, an oxazaphosphorine drug is a lymphocytotoxic but hematopoietic stem cell sparing high-dose pulsed amount of cyclophosphamide.

### III. Modes of Administration

The various compounds used in the methods described herein may be administered orally, parenterally (e.g., intravascularly such as intravenously), intramuscularly, sublingually, buccally, rectally, intranasally, intrabronchially, intrapulmonarily, intraperitoneally, topically, transdermally and subcutaneously, for example. Transplant cells may be administered to the subject by any effective route, such as orally, parenterally (e.g., intravascularly such as intravenously), intramuscularly, intracranially, intracerebrally, intradermally, intraocularly, nasally, topically, or by open surgical procedure.

The amount of compound or cells administered in a single dose may be dependent on the subject being treated, the subject's weight, the manner of administration and the judgment of the prescribing physician. Generally, however, administration and dosage and the duration of time for which a composition or transplant is administered will approximate that which is necessary to achieve a desired result.

Each dose of oxazaphosphorine drug may include an effective amount of an oxazaphosphorine drug in combination with a pharmaceutically acceptable carrier and, in addition, may include other medicinal agents, pharmaceutical agents, carriers, adjuvants, diluents, etc. Each dose of transplant cells may include an effective amount of transplant cells in combination with a pharmaceutically acceptable carrier and, in addition, may include other medicinal agents, pharmaceutical agents, carriers, adjuvants, diluents, etc. The optimal dosages for administration include those described herein and those, which may be routinely deter mined by a skilled artisan using well-known techniques.

The lymphocytotoxic, non-myeloablative amount of oxazaphosphorine drug is administered post-transplantation. However, in embodiments in which a pre-transplant conditioning regimen is employed, an oxazaphosphorine such as cyclophosphamide and/or one or more other immunosuppressive agents may also be administered to the subject before transplantation, such as fludarabine, anti-thymocyte globulin (ATG), pentastatin, 2-chlorodeoxyadenosine (2CdA), fludarabine-like drug, campath (alemtuzumab), busulfan, melphalan, chlorambucil, and uramustine.

In some embodiments, the lymphocytotoxic, non-myeloablative amount of oxazaphosphorine drug is in the range of about 40 mg/kg/day to about 60 mg/kg/day (e.g., about 50 mg/kg/day) for one or more consecutive days post-transplantation. Preferably, the lymphocytotoxic non-myeloablative amount of the oxazaphosphorine drug is administered for 1-4 consecutive days. In some embodiments, the lymphocytotoxic non-myeloablative amount of oxazaphosphorine drug is administered for two or more consecutive days. Preferably, the lymphocytotoxic non-myeloablative amount of oxazaphosphorine drug is administered starting 48-72 hours after transplantation, for 1-4 consecutive days. In some embodiments, the lymphocytotoxic non-myeloablative amount of oxazaphosphorine drug is administered starting at least 60 hours after transplantation. In some embodiments, the lymphocytotoxic non-myeloablative amount of oxazaphosphorine drug is administered starting within 72 hours after transplantation.

The dose for the oxazaphosphorine drug, e.g., cyclophosphamide, for use in the methods of the present invention can be calculated according to the ideal body weight of the subject. Ideal body weight can be determined, for example, according to Metropolitan Life tables, or any other standard known in the art. If the patient's actual body weight is less than ideal, the actual weight may be used for the calculation of the oxazaphosphorine drug dose.

In some embodiments, the present invention provides kits including one or more doses of high-dose pulsed amount of an oxazaphosphorine drug and cells intended for transplantation, packaged with instructions of use. Such instructions may pertain to use of the packaged components (i.e., one or more doses of a high-dose pulsed amount of an oxazaphosphorine drug and transplantation of the cells) in methods of reducing (treating, preventing, delaying onset of, ameliorating, or eliminating) a transplant rejection in a patient, by administering the one or more doses of high-dose lymphocytotoxic, non-myeloablative pulsed amount of an oxazaphosphorine drug after transplantation of the cells. The kit may further include one or more immunosuppressive agents and instructions for their administration (e.g., oxazaphosphorine, fludarabine, anti-thymocyte globulin (ATG), pentastatin, 2-chlorodeoxyadenosine (2CdA), fludarabine-like drug, campath (alemtuzumab), busulfan, melphalan, chlorambucil, and uramustine).

Depending on the intended mode of administration, the compounds used in the methods described herein may be in the form of solid, semi-solid or liquid dosage forms, such as, for example, tablets, suppositories, pills, capsules, powders, liquids, suspensions, lotions, creams, gels, or the like, preferably in unit dosage form suitable for single administration of a precise dosage. Each dose may include an effective amount of a compound used in the methods described herein in combination with a pharmaceutically acceptable carrier and, in addition, may include other medicinal agents, pharmaceutical agents, carriers, adjuvants, diluents, etc.

Liquid pharmaceutically administrable compositions or transplants can prepared, for example, by dissolving, dispersing, etc., a compound for use in the methods described herein and optional pharmaceutical adjuvants in an excipient, such as, for example, water, saline aqueous dextrose, glycerol, ethanol, and the like, to thereby form a solution or suspension. For solid compositions or transplants, conventional nontoxic solid carriers include, for example, pharmaceutical grades of mannitol, lactose, starch, magnesium stearate, sodium saccharin, talc, cellulose, glucose, sucrose, magnesium carbonate, and the like. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art; see, for example, Remington's Pharmaceutical Sciences, 18th Ed. (1990), Mack Publishing Co., Easton, Pa., the entire disclosure of which is hereby incorporated by reference).

### IV. Methods of Treatment

The method of the invention is a method for reducing (e.g., treating, preventing, or delaying the onset of) transplant rejection in a human or non-human mammalian subject, comprising administering a lymphocytotoxic non-myeloablative amount of a oxazaphosphorine drug to the subject, wherein the oxazaphosphorine drug is administered after transplantation.

In general, or HLA-haploidentical transplants, it is possible to administer relatively less oxazaphosphorine (e.g., cyclophosphamide) because the risk of graft rejection is reduced. In the case of a half-match, it is preferred to administer the oxazaphosphorine for two consecutive days before transplantation and for consecutive days after transplantation.

Optionally, in addition to administration of the lymphocytotoxic, non-myeloablative amount of oxazaphosphorine post-transplant, an oxazaphosphorine and/or other immunosuppressive agents or treatments may be administered pre-transplantation as a conditioning regimen. In some embodiments in which an oxazaphosphorine drug is administered post-transplant and (as part of a conditioning regimen) pre-transplant, the amount of oxazaphosphorine drug administered pre-transplant is typically less than the amount of oxazaphosphorine drug administered post-transplant. In other embodiments, the amount of oxazaphosphorine drug administered pre-transplant is about the same as the amount of oxazaphosphorine drug administered post-transplant. In some embodiments, the daily dose of oxazaphosphorine drug administered pre-transplant (e.g., 14.5 mg/kg body weight/day) is less than the daily dose of oxazaphosphorine drug administered post-transplant (e.g., 50 mg/kg body weight/day). In other embodiments, the daily dose of oxazaphosphorine drug administered pre-transplant is about the same as the daily dose of oxazaphosphorine drug administered post-transplant (e.g., 50 mg/kg body weight/day). Other methods of pre-transplant conditioning that are known in the art may be utilized (see, for example, Toze C L, Galal A, Barnett M J, et al (2005), Bone Marrow Transplant. 36(9):825-30; Alyea E P, Kim H T, Ho V, et al (2006) Biol. Blood Marrow Transplant. 12(10):1047-55; Alyea E P, Kim H T, Ho V, et al (2005) Blood 105(4):1810-4; Mielcarek M, Martin P J, Leisenring W, et al (2003) Blood 102(2):756-62; Alyea E P, Kim H T, Ho V, et al (2005) Blood 105(4):1810-4).

In preferred embodiments, anti-thymocyte globulin (ATG) is administered to the subject pre-transplant. Any pharmaceutically acceptable source of ATG may be utilized (e.g., rabbit, horse, etc.). In some embodiments, 10-40 mg/kg of ATG is administered pre-transplant over 2 to 4 days. In some embodiments, ATG is administered pre-transplant according to the following regimen: 0.5 mg/kg on day-9, 2 mg/kg on day-8, and 2 mg/kg on day-7. In some embodiments, 10-40 mg/kg of horse ATG is administered pre-transplant over 2 to 4 days. In some embodiments, rabbit ATG is administered pre-transplant according to the following regimen: 0.5 mg/kg on day-9, 2 mg/kg on day-8, and 2 mg/kg on day-7.

A subject in need of transplantation can be readily diagnosed based on methods well-known in the art. A subject having an autoimmune disorder can be readily diagnosed based on the methods well-known in the art and those described herein, e.g., by assaying for autoreactive antibodies.

The transplant rejection that is reduced using the methods of the invention may be a hyper-acute rejection, acute rejection, or chronic rejection. Examples of transplant rejections which may be reduced using methods described herein include, for example, transplant rejection occurring during or following allogeneic blood or marrow transplantation, or allogeneic transplantation of organs, tissues, or other cells into a host; transplant rejection occurring during or following a xenogenic transplantation of organs, tissues, or cells into a host; and transplant rejection occurring during or following transplantation of autologous tissue, organs or cells into a host. Transplant rejections also include rejections occurring during or following transplantation of an organ, tissue or hematopoietic stem cells from related (matched or partially matched) or unrelated donors. Transplant rejections that may be treated, delayed, or prevented with the invention include both graft rejection and graft-versus-host disease (acute or chronic). Without wishing to be bound by theory, it is contemplated that any transplant rejection which can be effectively reduced by eliminating the subject's circulating immune cells with high dose cyclophosphamide and allowing them to redevelop from hematopoietic stem cells is encompassed by the invention.

In one embodiment, the transplant cells are bone marrow cells (e.g., comprising bone marrow stem cells and/or other bone marrow cells) administered for treatment of a hereditary hemoglobinopathy such as sickle cell anemia and/or treatment of a hematologic malignancy. For example, the bone marrow transplant may be an allogeneic bone marrow transplant.

In some embodiments, the method for reducing transplant rejection further includes the step of administering by any effective route (e.g., orally, intravenously) an effective amount of mesna (2-mercaptoethan sodium sulfonate) or another agent that is effective in reducing, delaying the onset of, or preventing hematuria and/or hemorrhagic cystitis. In some embodiments, the method for treating, preventing, or delaying the onset of a transplant rejection further includes the step of administering an effective amount of a chemotherapeutic agent, such as fludarabine. In some embodiments, the method for treating, preventing, or delaying the onset of a transplant rejection further includes the step of administering an effective amount of an antimicrobial agent to the subject. In some embodiments, the method for treating, preventing, or delaying the onset of a transplant rejection further includes the step of administering an effective amount of granulocyte-colony stimulating factor (G-CSF) to the subject. In some embodiments, the method for treating, preventing, or delaying the onset of a transplant rejection further includes the step of administering an effective amount of platelets to the subject. In some embodiments, the method for treating, preventing, or delaying the onset of a transplant rejection further includes the step of administering an effective amount of red blood cells to the subject. The method for treating, preventing, or delaying the onset of a transplant rejection, as described herein, may include any one, two, three, four, five, or all six of these additional steps.

In some embodiments, a method encompassed by this disclosure includes a method for reducing transplant rejection, including administering a lymphocytotoxic non-myeloablative amount of a oxazaphosphorine drug to the subject, such that the subject's immune system reconstitutes without both stem cell transplantation and administration of additional immunomodulatory agents, and where the method does not include administration of platelets.

In some methods encompassed by this disclosure, an effective amount of granulocyte colony stimulating factor is 5 μg/kg/day, which is administered for a duration of time necessary for the neutrophil count to be at least 1000/mm³. In some embodiments, methods encompassed by this disclosure include administration of an effective amount of NEULASTA.

In some embodiments, a method for reducing a transplant rejection includes administering to a subject in need thereof, a lymphocytotoxic non-myeloablative amount of a oxazaphosphorine drug followed by, administering an effective amount of granulocyte colony stimulating factor to the subject; and administering an effective amount of at least one antimicrobial agent to the subject.

Mesna. In some embodiments, in addition to the oxazaphosphorine drug, mesna(2-mercaptoethan sodium sulfonate) or another agent is administered that is effective in reducing, delaying the onset of, or preventing hemorrhagic cystitis, which may potentially be induced by the oxazaphosphorine drug, such as amifostine or glutathione. Mesna is converted to a free thiol compound in the kidney, where it binds to and inactivates acrolein and other urotoxic metabolites of oxazaphosphorine drugs, to produce a non-toxic thioether and slows the rate of acrolein formation by combining with 4-hydroxy metabolites of oxazaphosphorines.

Dosing of mesna or a similar drug will depend upon the amount of oxazaphosphorine drug administered and the delivery route (e.g., oral, intravenous, etc.). For example, the total daily dose of mesna can be equal to about 80% of the total daily dose of oxazaphosphorine drug (e.g., cyclophosphamide). In some embodiments, such as in the case of treatment for sickle cell anemia, it is preferred that the mesna or similar drug is administered post-transplantation, immediately before and immediately after administration of a oxazaphosphorine drug (e.g., 30 minutes pre- and at 3, 6, and 8 or 9 hours post-oxazaphosphorine drug administration). In other embodiments, such as in the case of treatment of hematologic malignancies, it is preferred that the mesna or similar drug is administered pre- and post-transplantation, immediately before and immediately after administration of an oxazaphosphorine drug.

In one embodiment, 10 mg/kg (10 mg per kg of subject body weight) of mesna or a similar agent is given intravenously (iv) prior to oxazaphosphorine (e.g., cyclophosphamide) infusion, then 10 mg/kg iv at 3, 6, and 8 hours after the oxazaphosphorine administration, such that the total daily dose is 40 mg/kg. For example, for a 70 kg patient, the total dose of mesna over four days could be 11.2 grams

In addition to mesna and similar drugs, hydration treatment is preferably carried out.

Chemotherapy. In some embodiments, in addition to an oxazaphosphorine drug, a chemotherapeutic agent (other than an oxazaphosphorine drug), such as fludarabine or busulfan, is administered to the subject.

Fludarabine phosphate is a purine antimetabolite that, after administration, undergoes rapid conversion in plasma to the nucleoside 2-fluoro ara-A (F-ara). F-araA subsequently enters cells where it is phosphorylated to F-araATP and the monophosphate F-araAMP. Once activated, F-araATP inhibits DNA polymerase and ribonucleotide reductase. The monphosphate F-araAMP, once incorporated into DNA, is an effective DNA chain terminator.

Busulfan (1,4-dimethanesulfonoxybutane) is an alkylating agent. The drug is extensively metabolized and its metabolites are eventually excreted in the urine. The oral preparation is well absorbed.

In the case of sickle cell anemia, for example, the chemotherapeutic agent (e.g., fludarabine) can be administered prior to transplantation, e.g., days −6 to −2. In the case of a hematological malignancy, for example, the chemotherapeutic agent (e.g., busulfan), can be administered prior to transplantation, e.g., days −7 to −4, or days −6 to −3.

Infection Prophylaxis and Therapy. In some embodiments, a method of treating or delaying the onset of a transplant rejection additionally includes the step of administering an effective amount of at least one antimicrobial agent to the subject. Thus, in addition to an oxazaphosphorine drug, one or more antimicrobial agents is administered to the subject before, during, and/or after transplant. Preferably, infection prophylaxis is initiated pre-transplant, e.g., at day −6. However, antifungal prophylaxis is preferably initiated post-transplant, e.g., day +5.

Exemplary antimicrobial drugs that may be used in the methods described herein include, but are not limited to, Amdinocillin (Mecillinam), Amikacin, Amoxicillin, Ampicillin, Azithromycin, Aztreonam, Bacampicillin, Bacitracin, Carbenicillin indanyl sodium, Cefaclor, Cefadroxil, Cefamandole, Cefazolin, Cefdinir, Cefditoren, Cefepime, Cefixime, Cefinetazole, Cefonicid, Cefoperazone, Cefotaxime, Cefotetan, Cefoxitin, Cefpodoxime Proxetil, Cefprozil, Ceftazidime, Ceftibuten, Ceftizoxime, Ceftriaxone, Cefuroxime, Cefuroxime axetil, Cephalexin, Cephalothin, Cephapirin, Cephradine, Chloramphenicol, Cnnoxacin, Ciprofloxacin, Clarithromycin, Clindamycin, Cloxacillin, Colistimethate, Daptomycin, Demeclocycline, Dicloxacillin, Dirithromycin, Doxycycline, Enoxacin, Ertapenem, Erythromycin, Fosfomycin, Gatifloxacin, Gemifloxacin, Gentamicin, Grepafloxacin, Imipenem/Cilastatin, Kanamycin, Levofloxacin, Lincomycin, Linezolid, Lomefloxacin, Loracarbef, Mafenide, Meropenem, Methacycline, Methenamine mandelate, Methenamine hippurate, Methicillin, Metronidazole, Mezlocillin, Minocycline, Moxifloxacin, Mupirocin, Nafcillin, Nalidixic Acid, Neomycin, Netilmycin, Nitrofurantoin, Nitrofurazone, Norfloxacin, Novobiocin, Ofloxacin, Oxacillin, Oxytetracycline, Penicillin, Piperacillin, Polymyxin B, Rifamixin, Sparfloxacin, Spectinomycin, Streptomycin, Sulfadiazine, Sulfamethoxazole, Sulfisoxazole, Teicoplanin, Telithromycin, Tetracycline, Ticarcillin, Tobramycin, Trimethoprim, Trovafloxacin, Vancomycin, and a pharmaceutically acceptable salt or derivative thereof.

Exemplary combinations of antimicrobial agents include, but are not limited to, for example, Amoxicillin plus Clavulanate, Ticarcillin plus Clavulanic Acid, Trimethoprim plus Sulfamethoxazole, Piperacillin plus Tazobactam, Quinupristin plus Dalfopristin, and Ampicillin plus Sulbactam.

In certain embodiments, an antimicrobial agent is chosen from the group consisting of Amphotericin B, Amphotericin B Deoxycholate, Amphotericin B cholesteryl sulfate complex (ABCD), Amphotericin B lipid complex (ABLC), Amphotericin B liposomal, Caspofungin acetate, Clotrimazole, Fluconazole, Flucytosine, Griseo fulvin, Itraconazole, Ketoconazole, Miconazole, Nystatin, Pentamidine, Terbinafine, and Voriconazole.

In some embodiments, methods encompassed by this disclosure further include administration of an antiviral drug. Antiviral drugs include, but are not limited to, Abacavir, Aciclovir, Amantadine, CMV hyperimmune globulin (CYTOGAM®), Didanosine, Emtricitabine, Enfuvirtide, Entecavir, Ganciclovir, Lamivudine, Nevirapine, Ribavirin, Rimantidine, Stavudine, Valaciclovir, Vidarabine, Zalcitabine, and Zidovudine.

Immunomodulatory Agents. In some embodiments, the methods of the invention include administration of an immunomodulatory agent, such as an immunsuppressive agent, before, or during, or after administration of a oxazaphosphorine drug. In other embodiments, the methods of the invention exclude administration of an immunomodulatory agent, such as an immunosuppressive agent, before, or during, or after administration of a oxazaphosphorine drug.

In some embodiments, tacrolimus and/or mycophenolic acid mofetil (MMF) are administered to the subject to help prevent or reduce onset of transplant rejection (graft rejection and GVHD). Tacrolimus (also known as FK-506) is a macrolide immunosuppressant that inhibits lymphocytes by forming a complex with FKBP-12, calcium, and calmodulin, leading to the decrease in the phosphatase activity of calcineurin. Tacrolimus is well absorbed orally and can be used in combination with a oxazaphosphorine drug to treat, delay, or prevent transplant rejection, including organ rejection, such as liver, kidney, bone marrow, cardiac, pancreas, pancreatic islet cell, and small bowel transplant rejection.

Mycophenolate mofetil is an ester prodrug of the active immunosuppressant mycophenolic acid (MPA). This active metabolite is a noncompetitive, reversible inhibitor of inosine monophosphate dehydrogenase (IMPDH).

Preferably, tacrolimus and/or MMF is administered post-transplant (e.g., starting on day +5), and is administered under an appropriate dosage regimen for several days or weeks. In some embodiments, both tacrolimus and MMF are administered. For example, tacrolimus and MMF may be administered to the subject for up to about one year, and up to about five weeks, respectively.

Growth Factor Support. In some embodiments, a method for treating or delaying the onset of a transplant rejection further includes the step of administering an effective amount of granulocyte-colony stimulating factor (G-CSF), such as filgrastim (NEUPOGEN) or the polyethylene glycol form, pegfilgrastim (NEULASTA). In some embodiments, the amount of G-CSF administered is effective to achieve a neutrophil count of at least 500/mm³. In some embodiments, the amount of G-CSF administered is 5 μg/kg body weight/day. In some embodiments, the subject is administered 5 μg/kg body weight/day s.c. or i.v. starting at day +5 post-transplant and continuing until the ANC is greater than 1000 mm³ for three days or two consecutive measurements over a three day period. For use in the case of fungal infections or subsequent netropenia (ANC of less than 500/mm³), G-CSF can be continued until the WBC is greater than 10,000-15,000.

Transfusion Support. In certain embodiments, a method for treating, preventing, or delaying the onset of a transplant rejection additionally includes the step of administering an effective amount of platelets to the subject. In some embodiments, the amount of platelets is an amount effect to achieve a platelet count of at least 10,000 platelets/mm³.

In certain embodiments, a method for treating, preventing, or delaying the onset of a transplant rejection additionally includes the step of administering an effective amount of packed red blood cells (RBCs). In some embodiments, the amount of RBCs administered is sufficient to maintain a hematocrit level greater than 25%. In some embodiments, an effective amount of red blood cells are administered to a subject for a duration of time necessary for the hemoglobin to be maintained at least at 8.0 g/dl.

Radiation. In some embodiments, the method further comprises treating the subject with ionizing radiation (irradiating the subject) by partial or selective irradiation, or total body irradiation (TBI), total lymph node irradiation, radiolabeled antibody against leukocytes, etc. Preferably, irradiation is carried out pre-transplantation, e.g., about one day prior to transplantation (day −1), as part of pre-transplant conditioning. Administration of one or more immunosuppressive agents may be included in the conditioning regimen.

In further embodiments, this disclosure relates to a method of obtaining a cell population substantially free of cells capable of eliciting an adverse immune reaction to a transplant in a subject, including: (a) administering a lymphocytotoxic non-myeloablative amount of a oxazaphosphorine drug to the subject, followed by, (b) administering an effective amount of granulocyte colony stimulating factor to the subject; (c) administering an effective amount of at least one antimicrobial agent to the subject; and (d) administering an effective amount of platelets to the subject, where the method does not include the use of both stem cell transplantation and administration of additional immunomodulatory agents. Exemplary additional immunomodulatory agents include but are not limited to, for example, prednisone, cyclosporine, methotrexate, tacrolimus, pimecrolimus and azathioprine. The high dose cyclophosphamide therapy described herein is more effective than the low-dose therapy, which usually requires daily oral dosing or monthly intravenous pulses at 500-1000 mg/m² and has a higher risk of malignancies and premature menopause and/or infertility.

Re-immunization. Protective immunity to diseases preventable by routine vaccination may be lost following administration of an oxazaphosphorine drug and transplantation (e.g., following allogeneic or autologous blood or marrow transplantation), particularly in children. Therefore, reimmunization may be desirable at appropriate time intervals following transplantation to re-establish immunity, particularly with the traditional childhood vaccines. The re-immunization may involve passive immunization (administration of antibody-containing immunoglobulin preparations to provide temporary protection) and/or active immunization (administration of a vaccine, toxoid, or other immunogenic composition). Active immunization may involve administration of a live vaccine, inactivated, or component vaccine. Preferably, active immunization will involve an inactivated or component vaccine. Examples of diseases or infections against which it may be desirable to immunize or reimmunize the subject at an appropriate time post-transplantation (and post-oxazaphosphorine drug administration) include, but are not limited to, poliovirus, tetanus, diphtheria, measles, mumps, rubella, influenza, pneumonococcus, hepatitis B, hepatitis A, Haemophilis influenzae, Streptococcus pneumoniae, varicella zoster virus (shingles).

The specification is most thoroughly understood in light of the teachings of the references cited within the specification which are hereby incorporated by reference. The embodiments within the specification provide an illustration of embodiments in this disclosure and should not be construed to limit its scope. The skilled artisan readily recognizes that many other embodiments are encompassed by this invention. All publications and patents cited and sequences identified by accession or database reference numbers in this disclosure arc incorporated by reference in their entirety. To the extent that the material incorporated by reference contradicts or is inconsistent with the present specification, the present specification will supersede any such material, The citation of any references herein is not an admission that such references are prior art to the present disclosure.

The invention having been generally described, may be more readily understood by reference to the following examples, which are included merely for purposes of illustration of certain aspects and embodiments of the present invention, and are not intended to limit the invention in any way.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLE 1

### Exemplified Treatment Schema for Sickle Cell Anemia

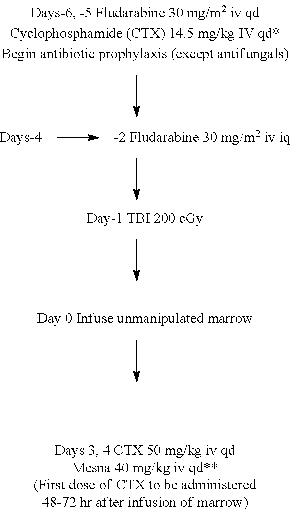

Placement of a double lumen central venous catheter can be used for administration of IV medications and transfusion of blood products. Preferably, documentation of a detailed history and physical examination and standard evaluation of cardiac, pulmonary, liver and renal function of the subject will be obtained.

Fludarabine can be administered by intravenous infusion over 30 min on D−6 to D−2. The dose will be 30 mg/ m². For decreased creatinine clearance (<61 ml/min) determined by the Cockcroft Formula:

CCr=(140−age)×IBW (kg)/PCr×72
x 0.85 (for women)
Fludarabine dosage should be reduced as follows:
CCr 46-60 ml/min, fludarabine=24 mg/m²
CCr 31-45 ml/min, fludarabine=22.5 mg/ m²
CCr 21-30 ml/min, fludarabine=19.5 mg/ m²
CCr <20 ml/min, fludarabine=15 mg/ m²

Cyclophosphamide can be administered as an iv infusion over 1-2 hours, (depending on volume) on D−6 and D−5. The dose of pre-transplantation cyclophosphamide is preferably about 14.5 mg/kg/day. Dose is calculated based on the adjusted ideal body weight or actual body weight whichever is less. Body weight and height can be measured directly. An approximate weight for height would be calculated from a standard table or equations that reflect ideal “values”.

Note: Hydration and Mesna can be utilized for the Day 3 and Day 4 post BMT cyclophosphamide doses, not for the pre-BMT cyclophosphamide doses.

Total body irradiation (TBI): 200 cGy AP/PA with 4MV or 6MV photons at 8-12 cGy/min at the point of prescription (average separation of measurements at mediastinum, abdomen, hips) can be administered in a single fraction on day −1.

Bone marrow can be harvested and infused on day 0. Donor bone marrow will be harvested with a target yield of 4×10⁸ nucleated cells/kg recipient IBW. Major incompatible ABO graft will have red blood cell depleted by huffy coat preparation. Minor ABO incompatible graft will have plasma removed.

Cyclophosphamide [50 mg/kg (LBW)] can be given on D+3 post-transplant (within 48-72 hr of marrow infusion) and on D+4 post-transplant. Cyclophosphamide (Cy) will be given as an iv infusion over 1-2 hr (depending on volume). Patients should be instructed to increase fluids overnight before cyclophosphamide administration. Hydration with normal saline at 3 cc/kg/hr i.v. will be started 2 hr prior to cyclophosphamide, then the rate will be reduced to 2 cc/kg/hr for 1 hr pre-cyclophosphamide and continued for 8 hr postcyclophosphamide or administered per institutional standards. Mesna will be given in divided doses i.v. 30 min pre- and at 3, 6, and 8 hr postcyclophosphamide or administered per institutional standards. Mesna dose will be based on the cyclophosphamide dose being given. The total daily dose of mesna is equal to 80% of the total daily dose of cyclophosphamide Preferably, no immuno-suppressive agents are administered to the subject until 24 hours after the completion of the post-transplant Cy. This includes steroids as anti-emetics.

### EXAMPLE 2

### Treatment Plan for HLA Matched Related and Unrelated Bone Marrow Transplantation With Busulfan/Cyclophosphamide and Post-Transplantation Cyclophosphamide for Hematological Malignancies

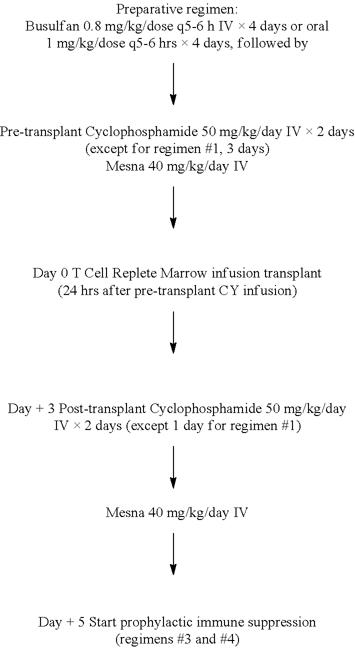

| | Busulfan | | | | |
|---|---|---|---|---|---|
| | 4.0 mg/kg/day oral | | | | |
| | divided q5-6h × 4 | | | | |
| | days OR 160 mg/m2/ | Pretransplant | Posttransplant | | |
| | day IV | Cyclophosphamide | Cyclophosphamide | FK506 | MMF |
| | divided q5-6h × 4 | 50 mg/kg/day | 50 mg/kg/day | 0.05 mg/kg IV | 15 mg/kg |
| Regimen # | days | IV | IV | or PO BID | PO TID |
| 1 | Busulfan d−7 to −4 | d−3 to −1 | d+3 | 0 | 0 |
| 2 | Busulfan d−6 to −3 | d−2 to −1 | d+3 to +4 | 0 | 0 |
| 3 | Busulfan d−6 to −3 | d−2 to −1 | d+3 to +4 | 0 | d+5 to +35 |
| 4 | Busulfan d−6 to −3 | d−2 to −1 | d+3 to +4 | d+5 to +50 | d+5 to +35 |

### Preparative Regimen Administration:

Dilantin dose will be based on weight per BMT unit standard of care for patients above 10 years of age.

Patients at least 6 years old receiving oral Busulfan will receive a starting dosage of 4 mg/kg/day PO divided Q5-6H for 4 days on days −6 through day −3 (days −7 through day −4 for regimen 1). (Note: For all regimens, Busulfan will be administered per standard of care for the BMT units. Busulfan doses extend into D−2 (or D−3 for regimen 1) secondary to the timing of dosing for kinetics). For patients under age 6, the starting dose will be 160 mg/m2/day, divided Q5-6H for 4 days. Patients will be made NPO 2 hrs before and after oral Busulfan to avoid interfering with the pharmacokinetic studies. Busulfan pharmacokinetics will be performed per standard of care for the BMT units.

Alternatively, IV Busulfex will be administered at a starting dosage of 3.2 mg/kg/day IV divided Q5-6H for 4 days for patients at least 6 years old, or 128 mg/m2/day IV divided Q5-6H for patients under age 6. Busulfex is diluted in 5% Dextrose or NS for IV infusion over 2 hours. For accurate pharmacokinetics, the IV tubing must be primed with drug, and connected as close as possible to the patient's central venous catheter. At the conclusion of the 2 hour infusion, the tubing must be disconnected so that no additional drug is given. With IV administration, blood samples will be drawn at 0, 60, 120, 125, 240, 359, and 360 minutes from the start of the first dose. Preference will be to use oral Busulfan due to ease of administration. However, the final decision about PO or IV formulation will generally be up to the attending physician.

Cyclophosphamide will be given at a dose of 50 mg/kg/day IV over 1 hr×2 days on day −2 and day −1 (50 mg/kg/day IV over 1 hr×3 days on days −3 through day −1 for regimen 1). Dosing of cyclophosphamide is based on ideal body weight for subjects whose ideal body weight is less than or equal to their actual body weight. On occasion, a subject's actual body weight may be less than his/her ideal body weight, in which case cyclophosphamide will be dosed using the subject's actual body weight. Intravenous hydration with appropriate fluids will be started at least 2 hr prior to cyclophosphamide and continued for at least 8 hr post-cyclophosphamide.

Mesna should be given to prevent hemorrhagic cystitis at 10 mg/kg/dose IV 30 min pre- and at 3, 6, and 8 or 9 hours post-cyclophosphamide. MESNA dose will be based on the cyclophosphamide dose being given. The total daily dose of MESNA is equal to 80% of the total daily dose of cyclophosphamide. Urine output over 2 hr will be checked before administering cyclophosphamide and must be at least 3.0 mL/kg. Urine output should be maintained postcyclophosphamide, as per BMT standards. Urinalysis will be performed to detect evidence of hemorrhagic cystitis, a known complication of high-dose Cy therapy. A day of rest may be added after the preparative regimen cyclophosphamide doses and prior to bone marrow infusion depending on donor availability, operating room schedules, and as clinically indicated.

Marrow will be infused on Day 0. Guidelines for the infusion of bone marrow have been established. The marrow infusion will be done by designated members of the BMT team. Preferably, the bone marrow graft will not be manipulated to deplete T cells. The donor will be harvested with a target yield of 3×10⁸ Nucleated cells/kg recipient IBW. Preferably, the lowest acceptable yield is 1.5×10⁸ Nucleated cells/kg. The CD 34+, CD4+, CD8+, and CD3+ cell count in the marrow can be quantified by flow cytometry.

### Post-Transplant Immunosuppression.

| | Busulfan | | | | |
|---|---|---|---|---|---|
| | 4.0 mg/kg/day | | | | |
| | oral divided q5- | | | | |
| | 6h × 4 days OR | Pre-transplant | Post-transplant | FK506 | |
| | 160 mg/m2/day | Cyclophosphamide | Cyclophosphamide | 0.05 mg/kg | MMF |
| | IV divided q5-6h × | 50 mg/kg/day | 50 mg/kg/day | IV or PO | 15 mg/kg |
| Regimen # | 4 days | IV | IV | BID | PO TID |
| 1 | Busulfan d−7 to −4 | d−3 to −1 | d+3 | 0 | 0 |
| 2 | Busulfan d−6 to −3 | d−2 to −1 | d+3 to +4 | 0 | 0 |
| 3 | Busulfan d−6 to −3 | d−2 to −1 | d+3 to +4 | 0 | d+5 to +35 |
| 4 | Busulfan d−6 to −3 | d−2 to −1 | d+3 to +4 | d+5 to +50 | d+5 to +35 |

No immuno-suppressive agents should be administered to the subject until 24 hours after completion of the post-transplant Cy.

### EXAMPLE 3

### Post-Transplantation High-Dose Cyclophosphamide (CY) is Effective Single Agent GVHD Prophylaxis that Permits Prompt Immune Reconstitution After Myeloablative HLA Matched Related And Unrelated Bone Marrow Transplantation (BMT) in Humans with Advanced Hematologic Malignancies

Prolonged pharmacologic immunosuppression is a major obstacle to early immunologic recovery after allogeneic BMT. Based on results in animal models, the inventors studied whether high-dose Cy alone after HLA-matched related or unrelated BMT is effective prophylaxis against severe acute GVHD while permitting effective reconstitution of lymphocytes, including regulatory T cells (T_{regs}). Forty-six consecutive patients (median age 41, range 1-64) with advanced hematologic malignancies received HLA-matched related (n=28) or unrelated (n=18) bone marrow after conditioning with busulfan on days −7 to −3 and Cy (50 mg/kg/day) on days −2 and −1, +3, and +4. No additional GVHD prophylaxis was administered. The cumulative incidence of acute grades II-IV and grades III-IV GVHD were 41% and 9%, respectively. Of the thirty-six patients alive after day 100, only 1 of the 23 patients that received HLA-matched related, and 3 of 13 patients that received unrelated allografts, developed chronic GVHD. With a median of 13 months (range 8-26 months) of follow-up, 26 (56%) patients are alive, of whom 21 (45%) are in complete remission. PBMCs collected at day 30-40, 40-60 and >60 post-transplant were evaluated enumerating CD4⁺ Foxp3⁺ cells by multi-color flow-cytometry and quantifying Foxp3, INF-γ, IL-2 and IL-10 mRNA transcripts by quantitative PCR. Patients with grade II-IV acute GVHD had significantly fewer CD4⁺Foxp3⁺ T cells compared to patients with grade 0-I GVHD (p<0.05). Development of grade II-IV GVHD negatively correlated with the expression of the Foxp3 (p<0.05) and was associated with marginally higher expression of INF-γ mRNA (p<0.08) suggesting higher effector function in the absence of Tregs in patients with grade II-IV GVHD. Expression of IL-2 mRNA transcripts was significantly higher in patients with grade II-IVGVHD compared to patients with grade 0-IGVHD (p<0.001). Differences in IL-10 mRNA expression was not observed. These results suggest that high-dose of post-transplantation Cy is effective as a single agent for limiting or preventing acute and chronic GVHD after HLA-matched related or unrelated BMT. This approach also limits the need for prolonged immunosuppression, resulting in favorable immunoreconstitution with few opportunistic infections in this unfavorable group of patients. The rapid recovery of T_{regs} associated with this strategy may also be responsible for the unexpectedly low incidence of chronic GVHD.

### EXAMPLE 4

### Reduced Intensity HLA-Haploidentical Bone Marrow Transplant With Post-Transplantation Cyclophosphamide in Humans with Non-Malignant Hematologic Diseases

Allogeneic blood or marrow transplantation (BMT) is potentially curative for a variety of life-threatening non-malignant hematologic diseases such as paroxysmal nocturnal hemoglobinuria (PNH) and hemoglobinopathies. The application of BMT to treat these disorders has been limited by the lack of suitable donors and often end-organ damage from the underlying disease.

Described herein are the results of three PNH patients, one of whom also suffered from sickle cell disease, treated with a reduced intensity allogeneic bone marrow transplant from an HLA-haploidentical donor using post-transplantation high-dose cyclophosphamide to mitigate graft-versus-host disease. The present inventors treated 3 patients with thrombotic PNH, one of whom also had sickle cell disease, with a non-myeloablative, HLA-haploidentical BMT with post transplant cyclophosphamide. Rapid engraftment without graft versus host disease occurred in 2 of the patients, including the patient with sickle cell disease. Both patients are disease free with full donor chimerism and require no immunosuppressive therapy, with follow-up of 1 and 4 years respectively. Non-myeloablative, HLA-haploidentical BMT with post transplant cyclophosphamide is a promising approach for patients with life-threatening non-malignant hematologic disease who lack an HLA-matched sibling donor.

Patient characteristics are listed in Table 1. Patients 1 and 2 had classical paroxysmal nocturnal hemoglobinuria (PNH) with multiple thromboses requiring thrombolytic therapy and systemic anticoagulation. Despite aggressive anticoagulation, both patients experienced progression of their thromboses leading to worsening performance status from Budd-Chiari syndrome. Patient 3 acquired PNH in the setting of hemoglobin SC disease and immune thrombocytopenic purpura. Before acquiring PNH, she experienced more than 10 pain crises a year that required hospitalization and 2-3 pain crises a month that she managed at home. Her immune thrombocytopenia was unresponsive to treatment with prednisone, intravenous immunoglobulin, splenectomy, rituximab and danazol. One year before her BMT she was diagnosed with PNH after presenting with multiple bouts of hemoglobinuria associated with back pain, abdominal pain and esophageal spasm distinct from her sickle cell pain. She required multiple red cell and platelet transfusions and developed a positive direct antiglobulin test and panreactive HLA antibody with a panel reactive antibody titer of 100.

TABLE 1
Patient characteristics

| | Age (yrs)/ | Duration of | PNH | | LDH | | | | Karnofsky |
|---|---|---|---|---|---|---|---|---|---|
| Patient | sex | PNH (years) | granulocytes | Sites of thrombosis | (U/L) | WBC | Hgb | Platelets | score |
| 1 | 27/male | 15 | 87% | hepatic v, portal v, | 2315 | 2120 | 7.8 | 73 | 50 |
| 2 | 37/male | 7 | 99% | saggital v, internal jugular v, | 709 | 4200 | 7.2 | 84,000 | 30 |
| | | | | hepatic v, pulmonary embolism, | | | | | |
| | | | | inferior vena cava | | | | | |
| 3 | 33/female | 1 | 50% | none | 1536 | 3860 | 10.7 | 6000 | 30 |

TABLE 2

| | | | | | | | | | | | Antigen | | Allele | |
|---|---|---|---|---|---|---|---|---|---|---|---|---|---|---|
| Patient/ | HLA Class I Alleles | | | | | | HLA Class II Alleles | | | | Level MM | | Level MM | |
| Donor | A1 | A2 | B1 | B2 | Cw1 | Cw2 | DRB1-1 | DRB1-2 | DQB1-1 | DQB1-2 | GVH | HVG | GVH | HVG |
| 1 | 0201 | 0201 | 0702 | 2705 | 0401/09N | 0102 | 0401 | 0701 | 0304 | 0303 | 3 | 3 | 4 | 5 |
| Donor- | 0201 | 3101 | 0702 | 4001 | 0401/09N | 0304 | 0401 | 0404 | 0304 | 0302 | | | | |
| Father | | | | | | | | | | | | | | |
| 2 | 3001 | 3201 | 1302 | 3801 | 0602 | 1203 | 0901 | 1301 | 0303/12 | 0603 | 4 | 4 | 5 | 5 |
| Donor- | 3001 | 2402 | 1302 | 3503 | 0602 | 0401/09N | 0901 | 1501 | 0303/12 | 0602/19 | | | | |
| Brother | | | | | | | | | | | | | | |
| 3 | 0301 | 6802 | 0702 | 0705/06 | 0702 | 1505 | 1501 | 1102 | 0602/19 | 0301/09 | 5 | 5 | 5 | 5 |
| Donor- | 0301 | 3001 | 0702 | 4201 | 0702 | 1701/02/03 | 1501 | 0302 | 0602/19 | 0402 | | | | |
| Mother | | | | | | | | | | | | | | |

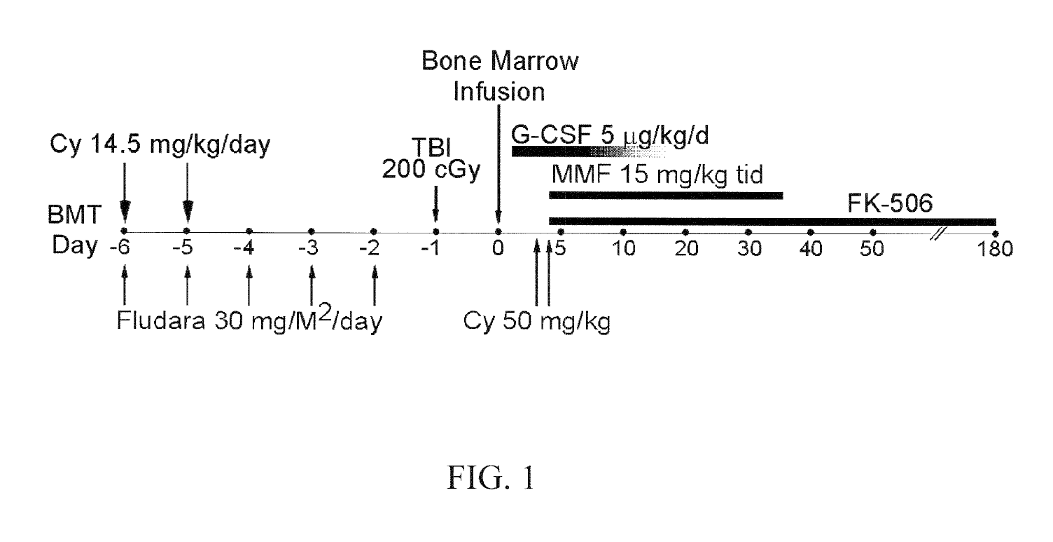

All three patients received conditioning therapy and post-transplant GVHD immunosuppression as previously described (O'Donnell, P. V. et al. (2006) Blood, 108, 894a-895a; O'Donnell, P. V. et al. (2002) Biol. Blood Marrow Transplant., 8, 377-386). Briefly, intravenous cyclophosphamide 14.5 mg/kg/day was administered on days −6 and −5, fludarabine 30 mg/m²/day IV on days −6 to −2 followed by 200 cGy of TBI on day −1 (treatment schema shown in FIG. 1). The marrow allograft infused on day 0 contained 1.38×10⁸ nucleated cells/kg (buffy coat prepared on a Gambro apheresis instrument for red cell depletion because of major ABO incompatibility), and 4.57×10⁶ CD34 positive cells/kg; 4.49×10⁸ nucleated cells/kg and 5.40×10⁶ CD34 positive cells/kg in patient 2; and contained 4.57×10⁸ nucleated cells/kg and 4.44×10⁶ CD34 positive cells/kg in patient 3. On days 3 and 4 post transplant, 50 mg/kg cyclophosphamide was administered over 90 minutes together with Mesna (80% of cyclophosphamide dose in 4 divided doses over 8 hours) by intravenous infusion. The patients received mycophenolate mofetil (CELLCEPT; Roche Laboratories, Nutley, N.J.) 15 mg/kg p.o. t.i.d. from day 4 to 35, and tacrolimus (PROGRAF; Fujisawa, Deerfield, Ill.) from day 4 to day 180 or 360. Tacrolimus was initiated at a dose of 1 mg IV daily, adjusted to achieve a therapeutic level of 5-15 ng/ml, and then converted to oral form until discontinuation. Filgrastim (NEUPOGEN, Amgen, Thousand Oaks, Calif.), 5 μg/kg/day was administered by subcutaneous injection starting on day 1 and continuing until recovery of neutrophils to >1000/μL for three days. Prophylactic anti-microbial therapy was started on day −6 and included norfloxacin 400 mg po twice daily, fluconazole 400 mg p.o. daily, appropriate prophylaxis for Pneumocystis carinii pneumonia, and valacyclovir, 500 mg p.o. thrice daily, as described previously. All patients were treated in the ambulatory transplant clinic.

At monthly intervals, nucleated cells were isolated from the marrow or peripheral blood or T cells (CD3-positive) and granulocytes (CD33-positive) were sorted from peripheral blood by flow cytometry. Percentages of donor-host chimeris for recipients of sex-mismatched BMT were determined by fluorescein in situ hybridization (FISH) using probes for X and Y chromosomes (Crescenzi B. et al., Cancer Genet. Cytogenet., 120:25-29 (2000)). For recipients of sex-matched BMT, chimerism was based on RFLP or PCR analysis of variable nucleotide tandem repeats unique to donors or recipients (Aaltonen L. A. et al., Science. 260:8912-816 (1993); Sreenan J. J. et al., Am. J. Clin. Pathol., 107:292-298 (1997); Van Deerlin V. M. et al., Clin. Lab. Med., 20:197-225 (2000)).

### Patient 1

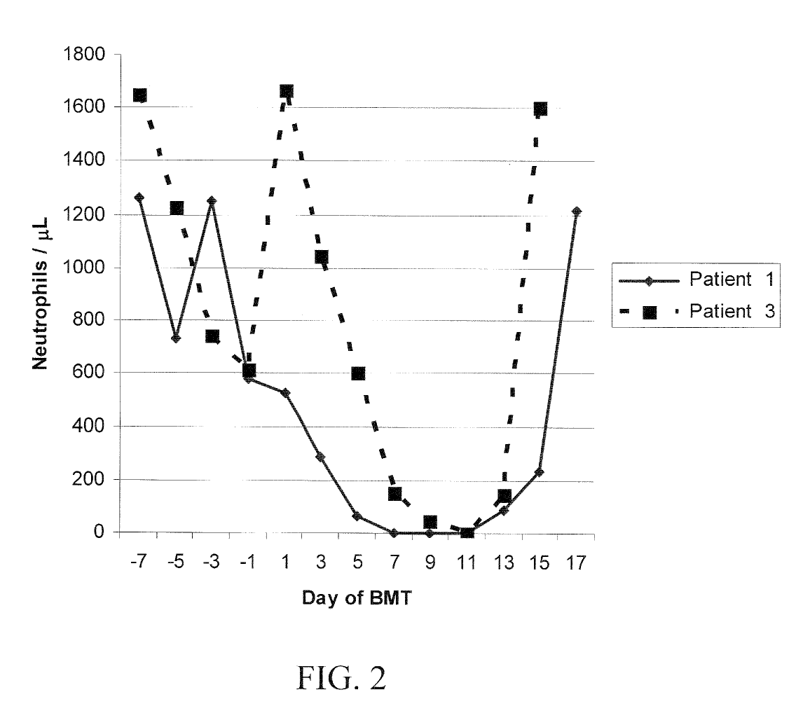

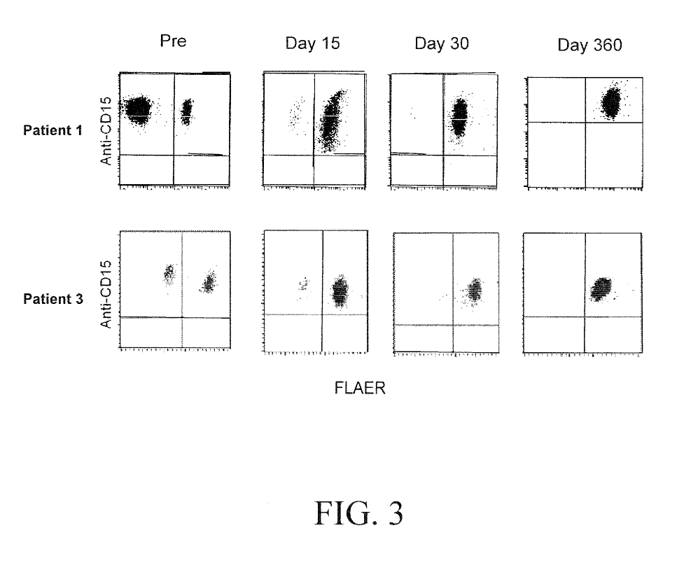

On day −1 through day five after BMT, the patient was admitted to the hospital for a PNH crisis that manifested with abdominal pain, hemoglobinuria, nausea, vomiting and fever lasting 6 days. He was readmitted for neutropenic fever on days 11 through 14. Blood cultures grew streptococcus viridans and he was treated with a 14-day course of vancomycin. The patient experienced rapid hematopoietic recovery (shown in FIG. 2). His absolute neutrophil count reached 500 μl on day 16 and he became transfusion independent of red cells and platelets on days 23 and 22, respectively. Full donor chimerism was documented on day 30 after BMT. By day 30, his PNH clone had regressed (shown in FIG. 3), his anticoagulation was discontinued, and all PNH manifestations had resolved. His tacrolimus was discontinued on day 360. The patient had no graft-versus-host-disease (GVHD). At 10 months post-transplant he developed varicella zoster of his right trigeminal nerve that resolved after treatment with acyclovir. He remains in a complete hematologic remission without evidence of PNH 48 months post-transplant.

### Patient 2

The patient was admitted to the hospital on day −4 of his transplant for candida krusei sepsis. Despite the use of broad spectrum antibacterial and antifungal antibiotics the patient developed multiorgan failure and expired from candida krusei sepsis 8 days after his HLA-haploidentical BMT. There was no evidence of engraftment at the time of death.

### Patient 3

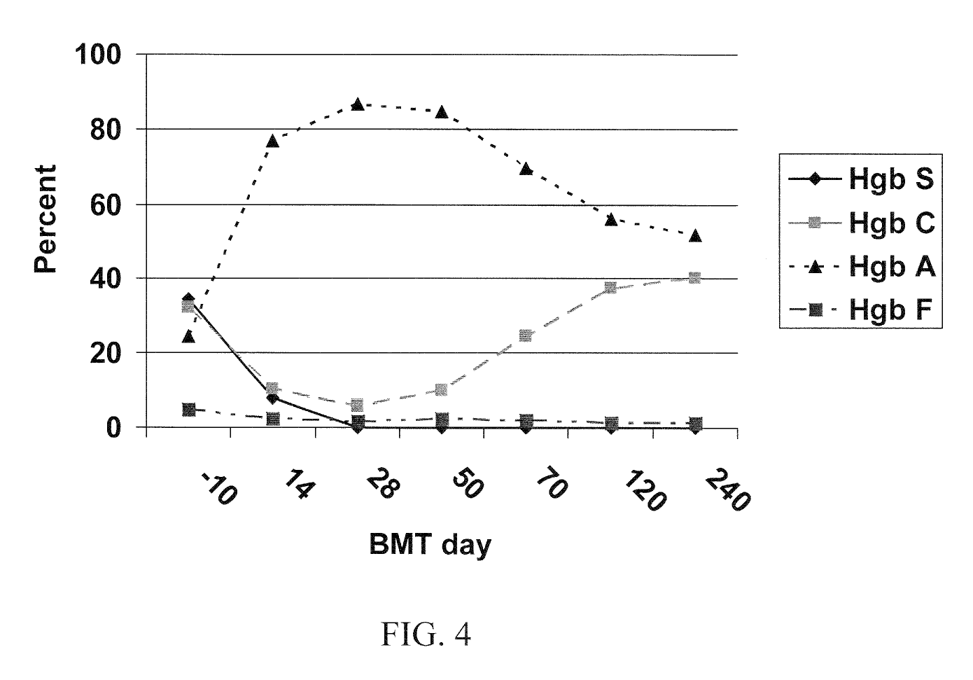

The patient was admitted for her conditioning regimen, due to pain from frequent sickle and. PNH crises and discharged 18 days after her BMT. She required patient controlled analgesia with hydromorphone and broad-spectrum antibiotics for febrile neutropenia. Her absolute neutrophil count reached 500 μl on day 14 and she became transfusion independent of red cells and platelets on days 26 and 17, respectively. Full donor chimerism was documented on day 30 after BMT. By day 30 after BMT, greater than 99% of her granulocytes were expressing GPI anchored proteins and by day 45 hemoglobin S was undetectable (shown in FIG. 3 and FIG. 4). Her tacrolimus was discontinued on day 180. She is now 1 year status-post BMT with no GVHD. Her RFLP shows no patient DNA and her sickle cell disease and PNH are in complete remission. Her donor was heterozygous for hemoglobin C. Accordingly, her most recent hemoglobin variant analysis reveals 52% hemoglobin A, 41% hemoglobin C and 1% hemoglobin F.

The present inventors used high-dose cyclophosphamide beginning three days after HLA-haploidentical BMT in 3 PNH patients (one of whom also had sickle cell disease) in an attempt to mitigate GVHD. In spite of the high level antigen mismatch between the hosts and donors, 2 of 3 patients, including the patient with sickle cell disease, achieved rapid hematopoietic engraftment with no GVHD. To the inventors’ knowledge, this is the first successful report of reduced intensity, HLA-haplo-identical BMT in patients with PNH or sickle cell disease.

All three patients in this trial were treated before eculizumab was commercially available; furthermore, none of these patients would have been eligible for the eculizumab trials. Now that eculizumab is FDA approved, the role of BMT in PNH may be decreasing (Hillmen, P. et al. (2006) N. Engl. J. Med., 355, 1233-1243; Brodsky, R. A. et al. (2008) Blood, 111, 1840-1847; Rother, R. P. et al. (2007) Nat. Biotechnol., 25, 1256-1264). Nevertheless, BMT offers the only potential for cure for PNH patients and may still be considered for those with life-threatening thrombosis.

The results described herein show that non-myeloablative, HLA-haploidentical BMT with post transplant cyclophosphamide can eradicate PNH. Furthermore, described herein is the first successful non-myeloablative HLA-haploidentical BMT in a patient with sickle cell disease. Reduced intensity HLA-haploidentical BMT with post-transplant cyclophosphamide can be administered to patients with compromised performance status and organ function. Two of three patients tolerated the procedure extremely well even though all three patients in this study had a Karnofsky performance status of 50 or below and significant end-organ disease. Moreover, there was no GVHD in the two evaluable patients suggesting that the post-transplant cyclophosphamide was effective in mitigating GVHD. Successful application of HLA-haploidentical BMT for hemoglobinopathies would greatly expand the pool of suitable donors offering a greater percentage of patients the prospect for cure.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or he able to ascertain using no more than routine experimentation, many equivalents to the specific embodiments of the invention described herein. Such equivalents are intended to be encompassed by the following claims.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A method for reducing transplant rejection in a mammal, comprising administering a lymphocytotoxic non-myeloablative amount of an oxazaphosphorine drug to the mammal, wherein the oxazaphosphorine drug is administered after transplantation.

2. The method of claim 1, wherein the transplant rejection is graft-versus-host disease (GVHD).

3. The method of claim 1, wherein the mammal is suffering from a malignant or non-malignant hematologic disorder, and the transplantation is carried out for treatment of the hematologic disorder.

4. The method of claim 3, wherein the hematologic disorder is sickle cell anemia, paroxysmal nocturnal hemoglobinuria (PNH), or a hematologic malignancy.

5. The method of claim 1, wherein the transplant comprises hematopoietic stem cells.

6. The method of claim 1, wherein the transplant is an allogeneic bone marrow transplant.

7. The method of claim 1, wherein the oxazaphosphorine drug is selected from the group consisting of: cyclophosphamide, ifosfamide, perfosfamide, trophosphamide, and a pharmaceutically acceptable salt, solvate, prodrug or active metabolite thereof.

8. The method of claim 1, wherein the lymphocytotoxic non-myeloablative amount of oxazaphosphorine drug is administered starting 48-72 hours after transplantation.

9. The method of claim 1, wherein the lymphocytotoxic non-myeloablative amount of oxazaphosphorine drug is administered starting at least 60 hours after transplantation.

10. The method claim 1, wherein the lymphocytotoxic non-myeloablative amount of oxazaphosphorine drug is administered on day+3 and day+4 after transplantation.

11. The method of claim 1, wherein the lymphocytotoxic non-myeloablative amount of oxazaphosphorine drug is in the range of about 40 mg/kg/day to about 60 mg/kg/day for 1-4 consecutive days.

12. The method of claim 1, wherein the lymphocytotoxic non-myeloablative amount of oxazaphosphorine drug is in the range of about 40 mg/kg/day to about 60 mg/kg/day for two or more consecutive days.

13. The method of claim 1, further comprising administering an effective amount of at least one of the following to the mammal before transplantation, after transplantation, or both before and after transplantation:
(a) a chemotherapeutic agent;
(b) an antimicrobial agent
(c) an anti-viral agent;
(d) an immunosuppressive agent;
(e) granulocyte-colony stimulating factor (G-CSF);
(t) platelets; or
(g) red blood cells.

14. The method of claim 1, further comprising carrying out pre-transplantation conditioning on the mammal.

15. The method of claim 14, wherein the conditioning comprises administration of an immunosuppressive agent.

16. The method of claim 15, wherein the immunosuppressive agent is selecting from the group consisting of an oxazaphosphorine, fludarabine, anti-thymocyte globulin (ATG), pentastatin, 2-chlorodeoxyadenosine (2CdA), fludarabine-like drug, campath (alemtuzumab), busulfan, melphalan, chlorambucil, and uramustine.

17. The method of claim 15, wherein the conditioning comprises administration of two or more immunosuppressive agents.

18. The method of claim 15, wherein the immunosuppressive agent comprises at east one alkylating agent.

19. The method of claim 18, wherein the alkylating agent is cyclophosphamide or a pharmaceutically acceptable salt or active metabolite thereof

20. The method of claim 14, wherein the conditioning comprises radiation treatment.

21. The method of claim 14, wherein the conditioning comprises administration of an immunosuppressive agent followed by treatment with total body irradiation.

22. The method of claim 14, wherein the conditioning comprises administration of cyclophosphamide and fludarabine, followed by treatment with total body irradiation.

23. The method of claim 14, wherein the conditioning comprises treatment with an immunosuppressive agent and anti-thymocyte globulin (ATG).

24. The method of claim 23, wherein 10-40 mg/kg ATG is administered over 2 to 4 consecutive days.

25. The method of claim 23, wherein the ATG is administered according to the following regimen: 0.5 mg/kg on day−9, 2 mg/kg on day—8, and 2 mg/kg on day−7.

26. The method of claim 1, further comprising administration of mycofenolate mofitil, tacrolimus, or both, after administration of the lymphocytotoxic non-myeloablative amount of an oxazaphosphorine drug.

27. The method of claim 1, further comprising re-immunizing the mammal to one or more pathogens after transplantation.

28. The method of claim 27, wherein said re-immunizing comprises administering an immunogenic composition that provides protective immunity to the mammal from one or more from among poliovirus, tetanus, diphtheria, measles, mumps, rubella, influenza, pneumonococcus, hepatitis B. hepatitis A, Haemophilis influenzae. Streptococcus pneumoniae, and shingles.

29. The method of claim 1, wherein the mammal is human.

30. A kit for treating or avoiding a transplant rejection including: (a) a plurality of doses of a lymphocytotoxic non-myeloablative but hematopoetic cell-sparing high-dose pulsed amount of a oxazaphosphorine drug; (b) cells for transplantation; and (c) instructions for reducing transplant rejection using one or more doses of the oxazaphosphorine drug post-transplant, wherein the transplant rejection is reduced.

31. The kit of claim 30, further comprising one or more immunosuppressive agents.

32. The kit of claim 31, wherein the one or more immunosuppressive agents are selected from the group consisting of an oxazaphosphorine, fludarabine, anti-thymocyte globulin (ATG), pentastatin, 2-chlorodeoxyadenosine (2CdA), fludarabine-like drug, campath (alemtuzumab), busulfan, melphalan, chlorambucil, and uramustine.

33. A conditioned transplant, comprising cells for transplantation that have been treated (in contact) with an oxazaphosphorine drug in vitro.

34. A composition comprising cells for transplantation and an oxazaphosphorine drug.
